(19) United States
(12) Patent Application Publication
(10) Pub. No.: US 2006/0088834 A1
(43) Pub. Date: Apr. 27, 2006

(54) Matrix analysis of gene expression in cells (magec)

(76) Inventors: Gretchen Bain, San Diego, CA (US); Lorrie Patricia Daggett, San Diego, CA (US); Mark Eben Massari, San Diego, CA (US); Xin Zhang, San Diego, CA (US)

Correspondence Address: MERCK AND CO., INC, P O BOX 2000, RAHWAY, NJ, 07065-0907, US

(21) Appl. No.: 10/510,722

(22) PCT Filed: Apr. 14, 2003
(86) PCT No.: PCT/US03/11581

§ 371 Date: May 20, 2005

Related U.S. Application Data
(60) Provisional application No. 60/372,476, filed on Apr. 15, 2002.

(51) Int. Cl. C12Q 1/68; C12N 15/87

(52) U.S. Cl. 435/6; 435/455

(57) ABSTRACT

The invention provides a novel expression cloning technique referred to as a matrix analysis of gene expression in cells (MAGEC) that allows for the indexed introduction, and analysis of nucleic acids in a host cell. While normally one takes cells attached to a surface followed by contacting the cells with heterologous DNA under conditions favoring the uptake of the heterologous DNA, the present invention, in sharp contrasts, proposes affixing (depositing) a nucleic acid-containing mixture onto a suitable surface and thereafter contacting suitable host cells (target cells) with the DNA-containing markings under conditions favoring uptake by the cells of the heterologous expression vector comprising a the target nucleic slide acid molecule. The method enables one to further characterize the gene product(s) of a known gene and unknown in a high-throughput assay format. It essentially allows for the identification of a gene based upon the function of its gene product.

### CROSS-REFERENCE TO RELATED APPLICATIONS

Not applicable.

### STATEMENT REGARDING FEDERALLY-SPONSORED R&D

Not applicable.

### REFERENCE TO MICROFICHE APPENDIX

Not applicable.

### BACKGROUND OF THE INVENTION

The present invention describes a novel assay that is suitable for high through-put screening. The herein disclosed methods, referred to as Matrix Analysis of Gene Expression in Cells (MAGEC), allow for the simultaneous transfection of a plurality of mammalian cells with known and/or unknown heterologous nucleic acid molecules. The method makes use of a gene expression matrix that has deposited thereon multiple expression constructs or nucleic acid molecules that are used to transfect host cells. The methods disclosed herein enable the identification of a particular nucleic acid based principally on a functional property of its gene product or its effects on the cell into which it was introduced. Methods of making a gene expression matrix as well as a transfected cell matrix are also provided. Applications to genetic screening, in vitro diagnostics, drug discovery etc are anticipated.

The emerging fields of proteomics, genomics and bioinformatics together with established recombinant DNA technologies are bringing powerful analytical capabilities to both research and clinical laboratories worldwide. Recent advances such as the sequencing and annotation of the human genome have yielded a wealth of data regarding the identity and classification of genes. However, sequence information alone is not always sufficient to infer the function of a given gene. Because of the inherent limitations of informatics tools, fully 42% of the genes identified in the human genome have no ascribed function (Venter et al. Science 2001 February 16; 291: 1304-1351). The field of functional genomics ultimately hopes to assign a function to all genes. Currently, however, methods for analyzing gene function (eg. expression cloning techniques) are performed on a gene-by-gene basis and are, by nature, low-throughput.

Although such methods for determining gene function have contributed immensely to our understanding of various disease states, they suffer from one or more disadvantages that render them unnecessarily inaccurate, time consuming, labor intensive, or expensive. Such disadvantages flow from requirements for, e.g., prior knowledge of gene sequences, cloning of complex mixtures of sequences into many individual samples each of a single sequence, repetitive sequencing of sample nucleic acids, electrophoretic separations of nucleic acid fragments, and so forth.

Likewise, conventional techniques for observing gene expression such as Northern blot analysis, RNase protection, or selective hybridization to arrayed cDNA libraries (see Sambrook et al., Molecular Cloning—A Laboratory Manual, Cold Spring Harbor Press, New York (1989)) depend on specific hybridization of a single oligonucleotide probe complementary to the known sequence of an individual molecule. Since a single human cell is estimated to express 10,000-30,000 genes (Liang et al., 1992, Science, 257:967-971), most of which remain uncharacterized, single probe methods to identify all sequences in a complex sample are prohibitively cumbersome and time consuming.

Moreover, traditional nucleic acid sequencing (Sanger et al., 1977, Proc. Natl. Acad. Sci. USA, 74:5463-5467), sequencing by hybridization (“SBH”) using combinatorial probe libraries (Drmanac et al., 1993, Science 260:1649-1652; U.S. Pat. No. 5,202,231, Apr. 13, 1993 to Drmanac et al.), or classification by oligomer sequence signatures (Lennon et al., 1991, Trends Genetics 7:314-317), and positional SBH (Broude et al., 1994, Proc. Natl. Acad. Sci. USA 91:3072-3076) all require purified clones making the methods inappropriate for complex mixtures.

As well, evaluating the function of cloned genes identified by conventional sequencing techniques generally entails cloning the discovered gene into an expression system suitable for functional screening. Transferring the discovered gene into a functional screening system requires additional expenditure of time and resources without guarantee that the correct screening system was chosen. Since the function of the discovered gene is often unknown or only surmised by inference to structurally related genes, the chosen screening system may not have any relationship to the biological function of the gene. For example, a gene may encode a protein that is structurally homologous to the beta-adrenergic receptor but have a dissimilar function. Further, if negative results are obtained in the screen, it can not be easily determined whether 1) the gene or gene product is not functioning properly in the screening assay or 2) the gene or gene product is directly or indirectly involved in the biological process being assayed by the screening system.

While several approaches have been described that attempt to characterize complex mixtures of nucleic acids without cloning, all of these require electrophoretic separation and/or traditional sequencing.

Differential display methods (Liang et al., 1992, Science 257:967-71; Liang et al., 1995, Curr. Op. Immunol. 7:274-280), which use the polymerase chain reaction (“PCR”) with an oligo (dT) primer and a degenerate primer designed to hybridize within a few hundred bases of the cDNA 3′-end, at best provide only qualitative “fingerprints” of gene expression, and suffer from well-known problems including a high false positive rate, migration of multiple nucleic acid species within a single observed band, and non-quantitative results.

Distinct from differential display is another class of methods, which observe gene expression by sampling, that is, these methods repetitively sequence nucleic acids in a sample and count the sequence occurrences in order to statistically observe gene expression. Such methods require sequencing and are statistically limited in their ability to discover rare transcripts. An early example of such a method sequenced and counted expressed sequence tags (“ESTs”) and determined frequency of expression of the ESTs (Adams et al., 1991, Science, 252:1651-1656). Another example is named “serial analysis of gene expression” (Velculescu et al., 1995, Science, 270:484-487). Approximate, putative identification of the source of a tag requires sequencing the tag and using the sequence and location information to look up possible source sequences in a nucleic acid sequence database.

In summary, conventional in vivo gene analysis can be done—on a gene-by-gene scale—by transfecting cells with a DNA construct that directs the overexpression of the gene product or inhibits its expression or function. The effects on cellular physiology of altering the level of a gene product is then detected using a variety of functional assays.

However, it is understood by those skilled in the art of recombinant DNA technology that conventional observational methods for gene expression monitoring are not capable of rapidly, accurately, and economically observing and measuring the presence or expression of selected individual genes or of whole genomes. More specifically, conventional gene expression methods have failed to provide a means for the high throughput screening of a plurality of genes in a timely and economic manner. These methods typically require, for example, prior knowledge of gene sequences, or cloning of complex mixtures of sequences into many individual samples of a single sequence, or repetitive sequencing of sample components, or electrophoretic separations, and so forth.

Over the last few years the human genome project has generated vast quantities of genes whose products need to be characterized. The robust physical properties of DNA have encouraged the creation of high-throughput screening tools to characterize both gene expression and function. Indeed, the vast quantities of DNA sequence data generated by various groups over the past decade has made use of standardized DNA microarrays or “chips” almost commonplace. Such chips have been used in studies ranging from gene expression monitoring and transcriptional profiling for drug target identification to large scale identification of single nucleotide polymorphisms (SNPs).

DNA arrays, consisting of thousands of DNA sequences printed at high density on a solid support, can be used for large-scale gene expression analysis (Ramsey, 1998, Nat. Biotechnol. 16:40-44; Marshall and Hodgson, 1998, Nat. Biotechnol. 16:27-31). Chips can be prepared by depositing already synthesized probe oligonucleotides on a derivatized glass surface, or by synthesizing the probe oligonucleotides directly on the glass surface using a combination of photolithography and oligonucleotide chemistry (Lashkari et al., 1997, Proc. Nat. Acad. Sci. USA 94:13057-13062; DeRisi et al., 1997, Science, 278:680-686; Wodicka et al., 1997, Nat. Biotechnol. 15:1359-1367; Chee et al., 1996, Science 274:610-614). These probe oligonucleotides are typically designed to hybridize to 10, 15, or 20 bases of a target DNA. The chips are hybridized to samples of fluorescently tagged target DNAs, and are then imaged to determine to which oligonucleotides hybridization has occurred. Total cDNA or mRNA samples can be used with this procedure, so that expression of thousands of genes in complex mixtures of cellular mRNA species can be simultaneously monitored (DeRisi et al., 1997, Science 278:680-686). This permits the detection within a defined cell population of characteristic transcript “signature” patterns which may be perturbed in characteristic ways by genetic mutations (DeRisi et al., supra) or manipulations of experimental conditions (DeRisi et al., supra; Wodicka, L., et al., 1997, Nat. Biotechnol. 15:1359-1367) thus suggesting that DNA microarrays may be useful for distinguishing desirable or undesirable effects during drug screening.

Although some success has been reported with such chips, well-known problems remain, including those of obtaining unambiguous and reliable hybridization signals. Typically, techniques to prepare labeled DNAs require isolation of the poly(A).sup.+ fraction of total cellular RNA, reverse transcription of mRNA by oligo dT-priming or random-priming, and labeling of cDNA by enzymatic or chemical methods. A large number of cells are necessary to produce the required amounts of mRNA. In addition, such labeling may alter the ability of the labeled polynucleotide to hybridize to the complementary sequence. Although DNA microarrays provide information about expression patterns within samples, they give no information about gene function.

Thus, existing techniques to prepare labeled samples are tedious, time-consuming and relatively insensitive. In several functional genomic approaches, large-scale functional characterization of gene products has become necessary. The fast pace of DNA recombinant technology has left a void in the art for high-throughput screening of defined nucleic acid molecules, without the need for prior sequencing of the target DNA etc. However, to be of broad benefit, gene expression techniques must allow for rapid, robust and precise induction/repression of gene activity. Thus, an assay format that would enable one skilled in the art to test hundreds to thousands of gene products for function in a single format instead of the conventional approach of testing a single DNA at a time, is an invaluable tool in studying gene expression and drug target validation. This need, in turn, has compelled investigators to identify prospective genes based upon a function of its gene product or so called “expression cloning”.

An early attempt at identifying genes based upon the identity of the function of the gene product of the cloned gene was reported in Nature by a group of investigators at MIT's Whitehead Institute. This group published a report detailing an experimental technique that uses whole cells expressing defined cDNAs as “probes” on microarrays. The methodology behind the transfected cell arrays noted supra proposes depositing onto a glass slide cDNA sequences, contained in expression plasmids and suspended in an aqueous gelatin solution. After drying, the slides are treated with a transfection reagent and placed in a dish. Adherent cells suspended in culture medium are then added. The cells settle, adhere to the slide, take in the expression plasmid and begin to express the gene of interest. Through cell division, each transfected cell results in a defined cluster of cells, each cluster expressing a single cDNA within a lawn of non-transfected cells. See U.S. Patent Publication No. 2002/0006664 A1 ('664 hereafter).

A key determinant of the proposed “reverse-transfection” technique described above is the use of a carrier protein, gelatin, and the mixing of the defined cDNA with gelatin (gelatin-DNA complex) or with gelatin and a lipid molecule (gelatin-lipid-DNA complex).

However, the disclosed “reverse transfection” method suffers from some drawbacks.
First—while the reverse-transfection method of Sabatini et al. ('664, supra) stresses the use of a “carrier” protein e.g., gelatin, to presumably facilitate transfection, the present invention, in sharp contrast does not utilize any carrier proteins but instead uses a non-proteinaceous polymer, such as glycogen or polyvinyl alcohol (PVA), to promote adherence of the transfection complex to the surface of the glass slide or plate.
Second—contrary to the statements contained in '664, supra, the “reverse transfection” method does not teach the use of multi-well plates for high-throughput gene-expression assays. For instance, '664 specifically exemplifies use of gamma amino propyl silane (GAPS) or poly-lysine coated glass slides and fails to enable use of a multi-well, coated plate(s). As such a plate-based high-throughput format is not enabled by the teachings of the “reverse transfection method”.
Third—as stated in '664, supra, the “reverse transfection” method utilizes gamma amino propyl silane (GAPS) or poly-lysine coated glass slides as the slide of choice, while the method of the invention prefers the use of Superfrost Plus positively charged slides.
Fourth—the methodology attending the “reverse transfection” method is limited to the use of lipid-based transfection reagents, thus limiting the scope of cell types that can be transfected and ultimately utilized for screening. The present invention makes use of non-lipid, polycationic transfection reagents e.g., polyethylenimine (PEI). These reagents have been previously shown to facilitate introduction of DNA into primary cells (eg. neurons) (Horbinski et al. BMC Neuroscience 2001 2:2, Dodds et al. J. Neurochem. 1999 72:2 105-2112, Boussif et al. Proc. Natl. Acad. Sci. USA 1995 92 7297-7301, Lambert et al. Mol. Cell. Neurosci. 1996 7 239-246). In addition, PEI has been shown to support the growth of primary neurons in culture dishes (Ruegg and Hefti, Neuroscience Letters 49 (1984) 319-324). Taken together, one skilled in the art would conclude that the aforementioned studies suggest that the use of PEI may indeed expand the scope of the proposed method by, for example, expanding the range of cell types that may be used for screening.
Fifth—it is unclear as to whether the methodology of the “reverse-transfection” method is suitable for transfecting cells/cell lines that are conventionally considered to be “less” transfectable because none of these so called “less” transfectable cell lines are described in '664, supra.

The present invention aims to overcome the above referenced drawbacks attending conventional gene expression monitoring methods.

The methods of the invention detail methodologies for screening nucleic acids in both slide and multi-well based platforms, thereby significantly expanding their scope.

As well, in certain aspects of the invention, the method disclosed herein does away with the requirement of a “carrier protein” and proposes instead the use of a non-lipid, polycationic transfection reagent e.g., polyethylenimine (PEI). Significantly, previous studies have demonstrated that PEI can be used to introduce DNA into primary cells (eg. neurons) (Horbinski et al. BMC Neuroscience 2001 2:2, Dodds et al. J. Neurochem. 1999 72:2 105-2112, Boussif et al. Proc. Natl. Acad. Sci. USA 1995 92 7297-7301, Lambert et al. Mol. Cell. Neurosci. 1996 7 239-246). In addition, PEI had been shown to support the growth of primary neurons in culture dishes (Ruegg and Hefti, Neuroscience Letters 49 (1984) 319-324). Taken together, one skilled in the art would conclude that the aforementioned studies suggest that the use of PEI may indeed expand the scope of the proposed method by, for example, expanding the range of cell types that may be used for screening.

Briefly, the present invention details a strategy for the cloning of a desired gene which is identified based upon a particular property (antigenicity, ligand binding, etc.) of the expressed gene. Conceivably, a cDNA library can be introduced into a suitable expression vector, e.g., eukaryotic expression vector and transfected into a large number of cells of various cell types to generate a gene expression matrix.

For example, a skilled artisan, seeking to identify a particular transport protein in accordance with a method of the invention, could conveniently use a labeled substrate on the gene expression matrix and identify cells expressing the target protein, e.g., transport protein by conventional techniques, e.g., autoradiography. Based upon the position of the signal within the matrix, the plasmid comprising the polynucleotide encoding the target protein could then be identified and the gene's sequence determined from the indexed matrix.

The herein disclosed gene expression matrix will also make it possible to perform hundreds to thousands of experiments in parallel. Thus, the ability to manage and manipulate the expression of multiple genes in parallel in a high throughput assay format analysis will further advance many areas of biology and medicine. Significantly, the methods disclosed herein will prove to be economical as well as less time consuming.

### SUMMARY OF THE INVENTION

The present invention provides a strategy for high throughput analysis of gene function in cells. One aspect of the present invention provides methods and reagents for creating an expression matrix-based system for analyzing gene expression in cells. The method of the invention is particularly suited for rapidly screening large sets of nucleic acids for those encoding desired products or for causing cellular phenotypes of interest.

Thus, for instance, a spatially defined gene expression matrix comprised of expression vectors containing nucleic acid molecules is used to generate a spatially defined matrix of transfected cells. The transfected cells can be screened for the ability of a transfected nucleic acid to confer a particular phenotype on the cell, and by reference to the position of the cell(s) on the matrix, the identity of the nucleic acid can be determined.

While particular embodiments of the invention are described in terms of DNA, it is to be understood that any suitable nucleic acid is encompassed by the present invention. Any suitable nucleic acid such as an oligonucleotide, DNA and RNA can be used in the methods of the present invention.

Importantly, the methods of the invention will also enable a skilled artisan to tailor gene expression to express target proteins, e.g., cell surface receptors or potential ligands including orphan receptors, which could be probed in an effort to identify potential binding partners in a shorter period of time, thereby effectively reducing the time needed to identify potential drug targets.

For instance, a transfected cell matrix, comprising cells transfected in accordance with a method of the invention can be probed with labeled binding partners, e.g., receptors or ligands. The mere location of the positive markings on the matrix will effectively lead to the identification of the nucleic acid of interest. This will also provide a crude measure of the affinity because several receptors, or variants of the same receptor, can be tested in parallel. Alternatively, other readouts such as calcium mobilization can be used to monitor activation of the receptors.

Importantly, the proposed methods of the invention are suitable for rapidly screening large sets of cDNAs or DNA constructs for those genes encoding desired products or causing cellular phenotypes of interest. Using slides/and or multi-well plates printed with expression vectors containing nucleic acids of interest, e.g. cDNAs, transfected cells matrices comprising cells expressing the respective gene products of the cDNAs can be constructed quickly and efficiently. The cell clusters can be screened for any property detectable within transfected cells and the identity of the responsible cDNA determined from the coordinates of the cell cluster with a phenotype of interest.

In furtherance of the above, a major application for the gene expression matrix disclosed herein is perceived to be receptor/ligand binding and functional studies. The small number of immobilized cells on the matrix is not a limitation to performing the above mentioned assays because signals can undergo amplification within cells and/or be further amplified by the detection systems. Signaling assays can be performed in several different formats using gene expression matrices of the invention. Activation of signaling pathways can be assessed by the use of antibodies that recognize activated forms of proteins. For example, gene expression matrices over-expressing hundreds of nucleic acids, e.g. cDNAs, can be treated with various stimuli followed by measurement of activation of a specific pathway (e.g. phosphorylation of a downstream kinase). In this way, molecules that inhibit or synergize with the stimulus can be identified.

As well, complementation studies can also be rapidly performed using the herein disclosed gene expression matrix. Indeed, a plethora of cDNAs for all candidate genes can be deposited on the support surface of a matrix and a cell line deficient for a given gene or function can be seeded on the resulting surface and the ability of the heterologous cDNA to rescue the phenotype can be monitored. Methods of monitoring are well known.

In accordance with the above, an embodiment of the present invention provides a method in which DNA (DNA of known or unknown sequence or source), also referred to as DNA of interest or heterologous DNA, is introduced into cells in defined areas of a lawn of eukaryotic cells, in which it will be expressed or will itself have an effect on or interact with a cellular component or function.

In the main, the method provides preparing a mixture comprising the DNA of interest (such as cDNA or genomic DNA) together with an adherence-promoting polymer such as glycogen or PVA and a suitable non-proteinaceous polycationic-based transfection reagent, such as PEI and appropriate amounts of an appropriate transfection enhancing-buffer, placing the same onto a suitable surface (e.g., a positively charged slide or other flat surface, such as the bottoms of wells in a multi-welled plate) in indexed locations. The polymer effectively promotes adherence of the DNA/transfection reagent to the desired substrate. The resulting mixture is then allowed to dry, thereby affixing the nucleic acid-containing mixture to the surface in defined distinct locations. Thereafter, suitable host cells are plated onto the surface under conditions favoring uptake of the nucleic acid by the host cells.

An alternative method follows the same steps as those recited immediately above except that a lipid-based transfection reagent is used in place of the non-proteinaceous, polycationic-based transfection reagent.

In the lipid-based embodiment, the nucleic acid is mixed with a adherence-promoting reagent such as glycogen or PVA and a lipid-based transfection reagent. The method comprises applying a nucleic acid-containing mixture (such as a cDNA, or genomic DNA or RNAi etc) which comprises a heterologous DNA together with appropriate amounts of an adherence-promoting polymer, and a lipid-based transfection reagent under conditions favoring the formation of a complex between the nucleic acid and the lipid-based transfection reagent, to a surface of a slide together with an appropriate buffer, and a suitable sugar, such as sucrose, thereby producing a surface bearing the nucleic acid/transfection reagent mixture in defined locations, wherein the adherence-promoting polymer facilitates adherence of the nucleic acid/lipid-based transfection reagent complex The resulting product is allowed to dry sufficiently such that the nucleic acid/transfection mixture is affixed to the surface and the nucleic acid-transfection complexes remain in the locations to which they have become affixed, under the conditions used for subsequent steps in the method. The glass slides are preferably Superfrost Plus positively charged glass slides and the multi-well plates are preferably poly-lysine-coated, polystyrene plates.

Accordingly, an aspect of the invention proposes introducing a nucleic acid of interest (target “nucleic acid” or “heterologous nucleic acid”) into cells in an indexed location within a monolayer of host cells, e.g. eukaryotic cells, in which the nucleic acid will be expressed and alter the function or cellular phenotype of the host cells. The method provides affixing a nucleic acid-containing mixture onto a surface of a suitable support medium, wherein the step of affixing comprises depositing a nucleic acid-containing mixture onto the surface of the support medium in defined distinct locations, wherein the nucleic acid containing mixture comprises heterologous nucleic acid that is intended to be introduced into suitable host cells in combination with appropriate amounts of an adherence-promoting polymer, and a polycationic transfection reagent, and allowing the nucleic acid-containing mixture to dry on the surface, followed by plating the host cells onto the surface of the support medium bearing the nucleic acid-containing mixture, under conditions favoring uptake of the nucleic acid by the host cells.

In the above embodiment, it is understood that a lipid-based transfection reagent can be substituted for the non-proteinaceous polycationic-based transfection reagent.

Use of the herein disclosed method(s) of introducing heterologous DNA into host cells in a high-throughput assay format is also an object of the present invention.

In an alternative embodiment, the transfected cells are maintained so as to facilitate the subsequent expression of the gene product encoded by the heterologous DNA.

In yet another embodiment, the invention provides maintaining the cells under conditions favoring expression of the heterologous DNA in the host cells.

In still another embodiment, the invention provides transfected cells comprising an expression vector, wherein the expression vector comprises a heterologous nucleic acid, wherein the cell is transfected in accordance with a method of the invention.

Methods for identifying transfected cells using a labeled probe are also provided. The method proposes introducing into the transfected host cell a labeled nucleotide probe having a nucleic acid sequence substantially complementary to that of the heterologous DNA under conditions favoring formation of a hybridization complex between the probe and the heterologous DNA, wherein presence of a complex is indicative of the successful transfection of the cells in accordance with at least one embodiment of the invention.

Another aspect of the invention provides a method for identifying host cells that expresses (transcribe and translate) a gene product encoded by the introduced heterologous nucleic acid. The method entails contacting the host cell with a labeled probe having binding affinity for the gene product of the heterologous DNA so as to form a complex there between. The identification of the complex, in turn, identifies the host cells expressing the gene product of interest.

A still further embodiment of the invention is drawn to affixing heterologous nucleic acids onto a suitable matrix by the steps described herein. The method for affixing heterologous nucleic acid molecule to a surface such as a slide or a well of a multi-well plate, to produce a matrix of heterologous nucleic acid in indexed locations of known or unknown sequence or source for use in a high throughput gene screening system, comprises applying a heterologous nucleic acid-containing mixture onto the surface in indexed locations. The resulting nucleic acid-containing mixture is allowed to dry sufficiently such that the mixture becomes affixed to the surface and remains affixed to the surface under conditions in which the matrix is used.

A still further embodiment is drawn to a method of depositing a heterologous DNA to a surface, to produce a micro-matrix or a macro-matrix by affixing a polymer-lipid-DNA mixture, as described herein, onto the surface in indexed locations (e.g., by hand or by using a robotic device, such as a micro arrayer). The resulting polymer-lipid-DNA mixture is allowed to dry sufficiently such that the mixture becomes affixed to the surface and remains affixed to the surface under conditions in which the matrix is used. This results in the production of a surface having affixed thereto lipid-DNA mixtures.

In an alternative embodiment, DNA is affixed to a surface, such as a well of a multi-well plate, to produce a matrix by affixing a linear polyethylenimine (PEI)-adherence-promoting polymer-DNA mixture, as described herein, onto the surface in indexed well locations (e.g., by hand or by using a robotic device, and allowing the resulting surface bearing the polymer-PEI-DNA mixture to dry sufficiently such that the mixture becomes affixed to the surface and remains affixed to the surface under the conditions in which the matrix is used. This results in the production of a surface having affixed thereto PEI-DNA mixtures.

A matrix comprising transfected cells is also provided.

A still further aspect of the invention is drawn to a method of producing a gene expression matrix in cells transfected with a heterologous nucleic acid comprising:

a) depositing a nucleic acid-containing mixture onto a surface of a suitable support medium in indexed locations and allowing the resulting surface bearing the nucleic acid-containing mixture to dry sufficiently that the mixture, referred to as nucleic acid-containing mixture, remains affixed to the surface under conditions in which the matrix is used, wherein the nucleic acid-containing markings include a heterologous nucleic acid in combination with a adherence-promoting polymer and a non-proteinaceous transfection reagent;

b) adding cells in an appropriate medium to the surface obtained in a) to produce a surface bearing heterologous nucleic acid-containing mixture and plated cells and

c) maintaining the surface bearing the heterologous nucleic acid and plated cells under conditions favoring the uptake of the heterologous nucleic acid by the plated cells, thus producing a matrix comprising transfected cells that contain the heterologous DNA.

With respect to cell surface receptors, e.g., G protein coupled receptors, once a cell has been identified as expressing a target receptor on its cell membrane, agonist or antagonist of this can then be identified. Indeed, an embodiment of the invention is drawn to a method useful for identifying DNAs whose expression alters (enhances or inhibits) a pathway, such as a signaling pathway in a cell or another property of a cell, such as its morphology or pattern of gene expression.

Thus, one aspect of the invention proposes a method for identifying an agonist or an antagonist of a cell surface receptor (target receptor), e.g., G protein coupled receptor (GPCR). The method comprises the steps of contacting the cell expressing the target receptor on its cell surface thereof with a candidate compound to be screened under conditions that permit binding of the compound to the receptor polypeptide, the receptor polypeptide being associated with a G protein that provides a detectable signal in response to the binding of the compound to the receptor polypeptide; and thereafter determining whether the compound binds to and activates or inhibits the receptor polypeptide by measuring the level of the signal generated from the interaction of the compound with the receptor polypeptide. Importantly, the cell expressing the GPCR is transfected in accordance with a method(s) of the invention.

The method may further comprise conducting the identification of the agonist or the antagonist in the presence of a ligand.

An alternative method for identifying an agonist or an antagonist of a G protein coupled receptor polypeptide comprises the steps of: determining the inhibition of binding of a ligand to cells that express the GPCR polypeptide on the surface thereof, or to cell membranes containing the GPCR polypeptide, in the presence of a candidate compound under conditions to permit binding of the compound to the GPCR polypeptide; and determining the amount of ligand bound to the receptor polypeptide, such that a compound that causes reduction of binding of the ligand is an agonist or an antagonist.

Also provided is a method of screening a compound for competitive binding to a target mammalian receptor on the surface of cells expressing the receptor, the method comprising the following steps: (a) transforming a host cell according to at least one method detailed herein for introducing and transforming cells with heterologous nucleic acid encoding said target receptor, wherein the transfected cells express the target receptor; (b) assaying the transfected cells with the compound in the presence and in the absence of an agonist for the target receptor; followed by (c) determining whether the compound competes with the agonist for binding to the target receptor.

As well, the invention provides screening for a compound to determine if the compound is an agonist binding inhibitor of a target mammalian receptor on the surface of cells expressing the receptor, the method comprising the following steps: (a) transforming a host cell according to at least one method of the invention with a heterologous nucleic acid encoding said target receptor, wherein the transfected cells express the target receptor; followed by b) assaying the transfected cells with the compound in the presence and in the absence of a target receptor agonist to determine whether the compound is capable of inhibiting agonist binding to the target receptor.

A method of screening a compound for binding a target mammalian receptor on the surface of cells expressing the receptor is also provided which contemplates (a) transforming a host cell according to a method of the invention with a heterologous nucleic acid encoding said target receptor, wherein the transfected cells express the target receptor; and (b) assaying the transfected cells with the compound to determine whether the compound binds to the target receptor.

Also the subject of the invention is a gene expression matrix or matrices of defined nucleic acids, e.g. cDNAs, affixed to a surface of the matrix. Such matrices can be produced by any of the methods detailed herein. Preferably, the methods are in accordance with either one of the two specific embodiments disclosed herein—adherence polymer-PEI-DNA embodiment or the polymer-lipid-DNA embodiment.

A transfected cell matrix, on the other hand, features cells that have taken up and expressed the encoded gene product, which is also provided.

Also provided is a method of expressing a nucleic acid of a known or unknown sequence or source, such as cDNA or genomic DNA, in defined locations or areas of a surface onto which different heterologous DNAs have been affixed, as described herein. Because each area of the surface has been affixed with DNA of known composition, it is a simple matter to identify the gene encoding the expressed protein.

An alternative embodiment proposes the use of the herein disclosed methods in identifying nucleic acids based upon the effect its gene product has on transfected host cells or how the DNA interacts with a cellular component(s) without being expressed, such as through hybridization to cellular nucleic acids or through antisense activity.

The present invention is described in the following Experimental Details Section, which is set forth to aid in an understanding of the invention, and should not be construed to limit in any way the invention as defined in the claims, which follow thereafter. Other features and advantages of the invention will be apparent to those of skill in the art upon further study of the specification and claims.

### BRIEF DESCRIPTION OF THE DRAWINGS

FIG. 1. Overview of MAGEC. Shown is a schematic representation of the MAGEC technology, which utilizes either a glass slide or multi-well tissue culture plate platform. A nucleic acid of interest is mixed with a transfection reagent and a non-proteinaceous polymer and deposited on the surface of a glass slide or to the bottom of a well of a multi-well plate and allowed to dry. A cell type of interest is then plated on the surface of the slide or multi-well plate to initiate transfection, thus creating a gene expression matrix. After the appropriate time has elapsed, any suitable assay may be performed on the gene expression matrix.

FIG. 2. Scan of emerald green fluorescence protein (EGFP) expression in HEK293 cells transfected by the MAGEC plated-based, PEI method.

FIG. 3. Effect of varying concentrations of compound (glutamate) on PI hydrolysis in HEK293 cells co-transfected by the MAGEC plate-based, PEI method with two GPCR nucleic acid molecules.

FIG. 4. Scans of emerald green fluorescence protein (EGFP) expression in HEK293, CHO K1 and N2A cells transfected by the MAGEC plated-based, lipid method.

FIG. 5. Effect of varying concentrations of compound (glutamate) on PI hydrolysis in HEK293 cells co-transfected by the MAGEC plate-based, lipid method with two GPCR nucleic acid molecules.

FIG. 6. Image of ³H-labeled drug binding to a cell surface protein. HEK293 cells transfected with a nucleic acid encoding a cell surface receptor, human calcium channel subunit—alpha2-delta-1.

FIG. 7. Western Blot analysis to detect human mGluR5 expressing cells using a human mGluR5 specific antibody. A slide-based MAGEC protocol was used to transfect the metabotropic glutamate receptor hmGluR5 into HEK cells. (A) through (D), the indicated cell type was used to seed glass slides printed with a mixture of a lipid-based transfection reagent, the hmGluR5 expression plasmid and an adherence-promoting polymer as described in Example 2. PVA, polyvinyl alcohol, PVP, polyvinylpyrrolidone, AP, amylopectin. In the field shown, nine spots (six for C), each about 1 mm in diameter, (˜20 nl of mixture) were deposited on the surface of the slides. Dark spots indicate cells within the matrix expressing hmGluR5.

FIG. 8. MAGEC-based intact cell-binding assay.

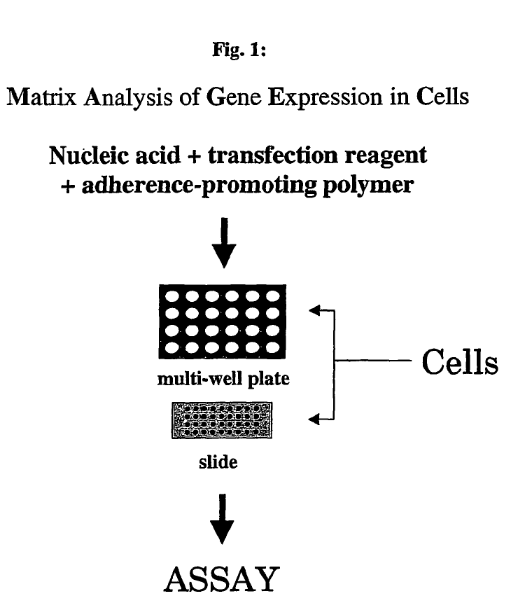

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

In the description that follows, a number of terms used in the field of recombinant DNA technology are extensively utilized. In order to provide a clearer and consistent understanding of the specification and claims, including the scope to be given such terms, the following definitions are provided.

As used herein and in the appended claims, the singular forms “an”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

The terms and abbreviations used in this document have their normal meanings unless otherwise designated. For example “N” refers to normal or normality; “mM” refers to millimole or millimoles; “g” refers to gram or grams; “ml” means milliliter or milliliters; “M” refers to molar or molarity; “μg” refers to microgram or micrograms; and “μl” refers to microliter or microliters.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

All publications mentioned herein are incorporated herein by reference for the purpose of describing and disclosing the methodologies, vectors etc which are reported in the publications that might be used in connection with the invention. Nothing herein is to be construed as an admission that the invention is not entitled to antedate such disclosure by virtue of prior invention.

Methods of the Invention

The methods of the present invention are drawn to the gene transfer of heterologous nucleic acid, e.g. DNA vector, into suitable host cells in a high throughput assay format. Matrices containing defined areas of known or unknown nucleic acid, contained in a suitable delivery vehicle are also a part of the invention as are transfected cells and matrices comprising the transfected cells. The invention also relates to gene transfer of a DNA vector and concomitant in vivo expression of the gene product encoded by the transfected DNA

Unlike traditional transfection techniques which rely on a prescribed sequence of events defined as attaching or affixing the host cell on a support medium followed by transfecting the cells with heterologous nucleic acid, the present invention proposes reversing the steps such that the target nucleic acid that is intended to be introduced into a host cell is attached to a medium and suitable host cells are plated thereon. Even though such a “reverse transfection” method has been proposed in U.S. Patent Publication No. 2002/0006664 A1 ('664) it will become apparent to one skilled in the art that the methods of the invention differ from those discussed in '664 supra as well as providing for a significantly broader spectrum of applications.

DNA/PEI Embodiment

In its most preferred embodiment, the present invention provides a gene-expression system for the functional analysis of numerous gene products in parallel.

In a first embodiment, the invention provides affixing a nucleic acid-containing mixture on a suitable support surface and letting the mixture dry. The mixture is formed by mixing or combining a nucleic acid of interest in an appropriate transfection enhancing buffer with a non-proteinaceous, polycationic-based transfection reagent and a non-proteinaceous adherence-promoting reagent and subsequently depositing the mixture onto a surface of a suitable support medium, e.g., delivered to the bottom of a well of a tissue culture plate.

After the nucleic acid-transfection reagent complex (e.g., a complex formed between the nucleic acid and the polycationic-based transfection reagent) has dried sufficiently that it is affixed to the surface of the support medium, actively growing cells are plated on top of the locations, thereby producing a surface that bears the nucleic acid-containing mixture in defined locations. The resulting dried product is maintained under conditions (e.g., temperature and time) favoring efflux of the DNA from the surface onto which is was previously affixed into the growing host cells. The appropriate conditions favoring uptake of the heterologous nucleic acid by the growing cells are well known and may vary according to the types of cells and reagents used and can be determined empirically. Temperature can be, for example, room temperature or 37° C. or any temperature determined to be appropriate for the cells and reagents.

Preferred adherence-promoting polymers include glycogen, polyvinyl alcohol, polyvinyl pyrrolidone (PVP), starch or methyl cellulose.

DNA/Lipid Embodiment

In the second embodiment, a mixture comprising a heterologous nucleic acid, (e.g. DNA contained in an expression vector) in an appropriate transfection enhancing buffer with an adherence-promoting polymer (e.g., polyvinyl alcohol); and a lipid-based transfection reagent is applied onto a surface, such as a glass slide, in indexed locations and allowed to dry. Thereafter, actively growing cells are plated on top of the nucleic acid-containing locations and the resulting surface is maintained under conditions (e.g., temperature and time) favoring entry of the nucleic acid contained in the nucleic acid-containing markings into the growing cells. The uptake and subsequent expression of the DNA in cells can be detected using known methods In an alternative format the surface is a well of a multi-well plate. Herein, the addition of an adherence-promoting polymer is not essential for efficient transfection. However, the addition of adherence reagents can promote better cell viability.

In practicing the method(s) of the invention, it is preferable that the host cells be actively growing eukaryotic cells into which the heterologous DNA is to be introduced and that such cells are placed on top of the surfaces onto which the DNA-containing mixture has been affixed. In general, as the cells grow, they take up the DNA, and make markings of localized transfection within a lawn of nontransfected cells. The resulting gene expression matrix which contains the dried transfection complex (be it a DNA/PEI complex or a DNA/lipid complex) and cells into which the heterologous DNA is to be introduced, are maintained under conditions appropriate for growth of the cells and entry of heterologous nucleic acid.

The cell bearing matrix that ultimately results (transfected cell matrix) is maintained in an appropriate medium, such as Dulbecco's Modified Eagles Medium (DMEM) containing 10% BCS with L-glutamine and penicillin/streptomycin (pen/strep). Other media can be used and their components can be determined based on the type of cells to be transfected. After a sufficient period of time, the transfected cells can be assayed for any desired function or activity associated with the transfected nucleic acid.

For example, the host cells may be engineered to express a reporter gene construct, which effectively allows one to monitor the level of expression of the target gene.

In the alternative, the host cells can be engineered such that a loss-of-function or gain-of-function phenotype is conferred to the transfected cells, thereby enabling one to assess a particular function by monitoring the ability of the members of the gene expression matrix to counteract the respective phenotype

The term “gene” as used herein is intended to refer to a nucleic acid sequence, which encodes a polypeptide. This definition includes various sequence polymorphisms, mutations, and/or sequence variants. The term “gene” is intended to include not only coding sequences but also regulatory regions such as promoters, enhancers, termination regions and similar untranslated nucleotide sequences. The term further includes all introns and other DNA sequences spliced from the mRNA transcript, along with variants resulting from alternative splice sites. A gene can be either RNA or DNA.

“Nucleic acid sequence” and its grammatical equivalents as used herein refers to an oligonucleotide, RNA, DNA, or any other nucleic acid molecule, and fragments or portions thereof, and to DNA of genomic or RNA of synthetic origin which may be single- or double-stranded, and represent the sense or antisense strand.

“Sequence” means the linear order in which monomers occur in a polymer, for example, the order of amino acids in a polypeptide or the order of nucleotides in a nucleic acid molecule.

As used herein, the term “heterologous nucleic acid” refers to a nucleic acid molecule, e.g., DNA or RNA, that does not occur naturally as part of the genome in which it is present or which is found in a location or locations in the genome that differs from that in which it occurs in nature. Heterologous DNA, which it is introduced into cells may be produced from other cells of the same or different type or synthetically produced. Examples of heterologous nucleic acid include, but are not limited to, DNA that encodes target polypeptides, receptors, reporter genes, transcriptional and translational regulatory sequences, selectable or traceable marker proteins, such as a protein that confers drug resistance. Examples of heterologous RNA include, but are not limited to, anti-sense RNA sequences, ribozymes, and double-stranded RNA (for inducing sequence-specific RNA interference).

An “indexed location” as used within the context of the present invention refers to deposition of nucleic acid mixtures in a distinct and defined position of the suitable support medium.

An “gene expression matrix” as used within the context of the present invention refers to a plurality of nucleic acids attached to one or more solid support surfaces where, when there is a multiplicity of support, each support bears a multiplicity of nucleic acids in indexed locations (markings). The term “matrix” can refer to the entire collection of nucleic acids on the support(s) or to a subset thereof. The DNA-containing mixture can be deposited in as many indexed locations as desired. A matrix can be made over a wide range of sizes depending upon the need of the skilled artisan. Techniques for depositing heterologous nucleic acids onto a surface are well known. The numerous ways in which the heterologous nucleic acid can be arranged on a matrix are also well known. The surface of the matrix may be rigid or flexible as the need arises.

A “gene expression matrix library” is a library of expression constructs prepared from genetic material derived from one or more species of donor organisms, and cloned in a suitable delivery vehicle, e.g., a vector that enables the expression of the genetic materials into host cells.

As used herein, the terms “target nucleic acid” and “nucleic acid of interest” refers to a component of a gene expression matrix, e.g., the portion or portions of a nucleic acid being transfected into host cells, which is of interest with respect to its ability to confer a change in the phenotype of the host cells. In general, though not always, the target nucleic acid will be that portion(s) of the nucleic acid of the gene expression matrix that is varied from one portion of the matrix to the next. The target nucleic acid can be a coding sequence for a protein, a “coding” sequence for an RNA molecule (e.g., which is transcribed into an anti-sense RNA sequence, a ribozyme or double-stranded RNA), or a regulatory sequence (e.g., as part of a reporter construct), to name but a few examples.

As used herein, “gene product of interest” refers to any protein encoded by the heterologous nucleic acid that, when expressed in a host cell, confers upon the host cell a desired characteristic.

The term “loss-of-function”, as it refers to the effect of a heterologous nucleic acid molecule, refers to those nucleic acid sequences which, when expressed in a host cell, inhibit expression of a gene or otherwise render the gene product thereof to have substantially reduced activity, or preferably no activity relative to one or more functions of the corresponding wild-type gene product.

As used herein, a “desired phenotype” refers to a particular phenotype for that the user of the subject method seeks to have selectively conferred on the host cell line upon expression of a target sequence.

The terms “protein”, “polypeptide” and “peptide” are used interchangeably herein.

As used herein, “cell surface receptor” refers to molecules that occur on the surface of cells, interact with the extracellular environment, and transmit or transduce the information regarding the environment intracellularly in a manner that may modulate intracellular second messenger activities or transcription of specific promoters, resulting in transcription of specific genes.

As used herein, “extracellular signals” include a molecule or a change in the environment that is transduced intracellularly via cell surface proteins that interact, directly or indirectly, with the signal. An extracellular signal or effector molecule includes any compound or substance that in some manner alters the activity of a cell surface protein. Examples of such signals include, but are not limited to, molecules such as acetylcholine, growth factors and hormones, lipids, sugars and nucleotides that bind to cell surface and/or intracellular receptors and ion channels and modulate the activity of such receptors and channels. The term also include as yet unidentified substances that modulate the activity of a cellular receptor, and thereby influence intracellular functions. Such extracellular signals are potential pharmacological agents that may be used to treat specific diseases by modulating the activity of specific cell surface receptors. “Orphan receptors” is a designation given to a receptors for which no specific natural ligand has been described and/or for which no function has been determined.

As used herein, a “reporter gene construct” is a nucleic acid construct that includes a “reporter gene” operatively linked to at least one transcriptional regulatory sequence. Transcription of the reporter gene is controlled by these sequences to which they are linked. The activity of at least one or more of these control sequences is directly or indirectly regulated by the target receptor protein. Exemplary transcriptional control sequences are promoter sequences. A reporter gene is meant to include a promoter-reporter gene construct, which is heterologously expressed in a cell.

The term “modulation of a signal transduction activity of a receptor protein” in its various grammatical forms, as used herein, designates induction and/or potentiation, as well as inhibition of one or more signal transduction pathways downstream of a receptor.

As used herein, the term and “transfected host cell” refers to a cell that includes a heterologous nucleic acid molecule, wherein the cell is transfected in accordance with the methods of the invention. Host cells contemplated for use in the practice of the present invention include cells well-known in the art. As used herein, the phrase “host cell” refers to both prokaryotic and eukaryotic cells, such as mammalian cells (e.g., HEK 293, CHO and Ltk.sup.-cells), yeast cells (e.g., S. cerevisiae, Candida tropicalis, Hansenula polymorpha, Pichia pastoris (see U.S. Pat. Nos. 4,882,279, 4,837,148, 4,929,555 and 4,855,231), and the like), bacterial cells (e.g., Escherichia coli), insect cells, and the like.

The introduced heterologous nucleic acid may be from the same species as the host cell or of a different species from the host cell, or it may be a hybrid DNA sequence, containing some foreign and some homologous DNA.

By “comprising” it is meant including, but not limited to, whatever follows the word “comprising”. Thus use of the term indicates that the listed elements are required, but that other elements are optional and may or may not be present.

By “consisting essentially of” is meant that the listed elements are required, but that other elements are optional and may or may not be present depending upon whether or not they affect the activity or action of the listed elements.

Although for simplicity this disclosure often makes references to single cell (e.g., “method of transfection a host cell”), it will be understood by those of skill in the art that more often any particular step of the invention will be carried out using a plurality of genetically similar cells, e.g., from a cultured cell line. Such similar cells are called herein a “cell type”. Such cells are derived either from naturally single celled organisms, or derived from multi-cellular higher organisms (e.g., human cell lines).

Recombinant DNA technology refers generally to techniques of integrating genetic information from a donor source into vectors for subsequent processing, such as through introduction into a host, whereby the transferred genetic information is copied and/or expressed in the new environment. Commonly, the genetic information exists in the form of complementary DNA (cDNA) derived from messenger RNA (mRNA) coding for a desired polypeptide product. The carrier is frequently a plasmid having the capacity to incorporate cDNA for later replication and/or expression in a host and, in some cases, actually to control expression of the cDNA and thereby direct synthesis of the encoded product in the host.

Transient transfection techniques, in which known cDNAs are transiently transfected into a variety of cell-types that have been previously plated on tissue culture dishes, are well established cell culture protocols that have been routinely used by biologists for more than 25 years. The use of these techniques has been invaluable in identifying the function of individual cDNAs or collection of cDNAs.

The recent identification of lipid-based transfection reagents has helped to increase transfection efficiencies in some cell types. However, limitations of these transient transfection protocols still exist. Notwithstanding the benefits afforded by these lipid-based transfection reagents, conventional transfection techniques are encumbered by numerous limitations, which include, but are not limited to:

i) The protocols are very labor intensive and costly to perform even if one were to screen a small number of variants (<6) of a particular cDNA or collection of cDNAs.

ii) The basic protocols do not allow for screens of large numbers of cDNAs in a slide array format since the cells are plated first and spatial deposition of the cDNAs is impossible post plating of the cells to the slide. While screening of multiple variants may be possible in a multi-well format, the labor-intensive nature of the procedure and the reproducibility of the results is a significant limitation.

iii) It would be virtually impossible to perform functional screens on large cDNA libraries or collections of cDNAs using conventional transfection protocols. The time and reagent costs is very prohibitive. As well, there are problems associated with consistent treatment of samples and reproducibility of samples due to the large number of manipulations required in the procedures that are not very amenable to robotic intervention.

The methods described herein aim to alleviate or eliminate the above referenced limitations. For example, the methods of the invention are well suited for the high-throughput transfection of mammalian cell lines—high throughput functional drug screening of cDNA-encoded target proteins in mammalian cells. In addition, the proposed methods of the invention are suited for both tissue culture plates and slide matrix formats because the nucleic acids, e.g. cDNAs, are deposited onto the slide or plate before the cells are added. Conceivably, robotic arrayers or pipettors are capable of depositing thousands of cDNAs in defined locations very quickly and efficiently in both formats. The robots can also be used to add the cells to the slides or plates, which further streamlines the procedure. The defined placement of DNAs before the cells are plated, in time, allows for very large screens with known or unknown cDNA libraries since once the assay has been performed it will be very simple to determine which cell(s) have responded in the matrix and therefore determine which cDNA or collection of cDNAs interacted with the drug or compound utilized in the screen. The proposed matrix based methods of the invention can be performed in a much shorter time frame and with significantly reduced labor costs and a higher degree of reproducibility. It is known that many receptors are natively expressed as multimeric subunits or require functional expression of auxiliary subunits. Since a large number of duplicate gene expression matrices can be produced with the MAGEC protocol, multiple screens of the same cDNA collection can be performed with a variety of cell types to determine if a particular host cell imparts either a more native phenotypic expression or if the cell type provides a subunit(s) required for function of the deposited nucleic acid.

Other proposed uses for the claimed methods include (a) screening cDNA collections or libraries for off-target activities or toxicological properties associated with known drugs, (b) identifying the molecular targets of known drugs by screening cDNA collections or libraries or genomic DNA fragments or libraries in order to isolate proteins that bind the drug of interest (reverse pharmacology), (c) identifying molecular targets of known drugs by screening cDNA libraries and thereby isolating proteins, which exhibit a functional response to the drug of interest (reverse pharmacology); (d) using the proposed methods of the invention to identify a function or activity of interest wherein the heterologous nucleic acid molecule is used as an antisense construct, or functions as a RNAi probes (inverse genomics); and (e) identifying proteins of interest through expression cloning in mammalian cells using assays based principally on function, reporter gene activity or antibody reactivity.

An important aspect of the method of invention is that the optimal concentration of heterologous DNA transfected using the method described herein will vary depending on the cell type, the nucleic acid being expressed and the regulatory element driving expression of the heterologous DNA. The optimal concentration of nucleic acid for use in the methods of the invention can range from 20 ng to 4 μg per well of a 96-well plate and must be determined empirically for each cell type and nucleic acid being transfected by the detailed methods described herein

Another important aspect of the detailed methods is that the effects on cellular physiology by the gene product of the heterologous DNA can be detected using conventional techniques. Rather than having to recover the transfected construct to ascertain its identity, the identity is determined by the position of the transfectant of interest on the matrix. A desired gene is essentially identified based upon the function of its gene product and its effect on the cell in which it is expressed.

Significantly, the methods of the invention provide a flexible assay platform and can support high-throughput functional screening of nucleic acids including libraries or collections of cDNAs, antisense oligonucleotides, double stranded RNAi oligonucleotides or genomic DNA fragments.

It is preferable to use eukaryotic cells (in an appropriate medium) in the method of the invention, i.e., suitable eukaryotic cells are plated on top of the dried DNA-containing markings at a density favoring the uptake of the DNA by the host cells. Ultimately, the heterologous DNA in the DNA-containing mixture enters the host cells and its gene product is expressed therein. Importantly, the proposed method of the invention provides not only a high transfection efficiency but it may also be used to transfect cells or cell types conventionally known to be “less transfectable.”

For use in the methods of the invention, the heterologous nucleic acid may be obtained from any source. For example, it can be genomic DNA or synthetic DNA or RNA including messenger RNA and RNAi.

It is preferable that the heterologous nucleic acid be substantially pure, most preferably it should be isolated in pure form from its source and be free of contaminants that are generally associated therewith. Use of the terms “isolated” and/or “purified” in the present specification as a modifier of nucleic acid, DNA, or RNA, means that the DNA, RNA, so designated have been produced in such form by the hand of man, and thus are separated from their native in vivo cellular environment.

All or a portion of the heterologous nucleic acid sequence can be synthesized chemically in accordance with techniques well known to a skilled artisan. Modifications to the synthetic nucleic acid may be introduced using well known techniques.

The nucleic acid for use in the methods of the invention can be linear or circular, double stranded or single stranded, and of any size. Preferably, the nucleic acid is double stranded and of sufficient length that facilitates uptake by the host cell without being degraded. The heterologous nucleic acid that can also be a DNA-RNA hybrid. The length of the nucleic acid for use as the heterologous nucleic acid may vary from 20 to 50,000 base pairs. In general, the length of the nucleic acid must be such that it imparts a distinct phenotype on the host cell.

Where the nucleic acid for use in the methods of the invention is present in a vector that is present in a cell, such material may be obtained by culturing the cell and extracting the nucleic acid from the cell by methods known in the art.

Alternatively, the nucleic acid can be obtained in small quantities and amplified using known techniques, e.g., amplification using the polymerase chain reaction (PCR). Refer to U.S. Pat. Nos. 4,683,202 and 4,683,195, which are incorporated by reference in their entirety.

If using nucleic acids isolated from a natural source, such source can be any cell or collection of cells. Adult tissue or embryonic tissue or organs at any given developmental stage are representative examples of the source of natural nucleic acid for use as the starting material in the methods of the invention.

In cases where the nucleic acid for use as the starting heterologous nucleic acid in the methods of the invention is a bodily fluid, (e.g. blood, lymph and other bodily fluid), the cells can be isolated from the fluid by such techniques as filtration, affinity purification, centrifugation etc. Techniques for isolating cells are well known to those skilled in the art.

Where the heterologous nucleic acid is synthetic DNA, e.g., cDNA that has been derived from mRNA isolated from a target source, the mRNA may be isolated using a variety of methods well known to a skilled artisan. See U.S. Pat. No. 4,843,155.

Where the nucleic acid for use as the starting material in the method of the invention is derived from genomic DNA, techniques for isolating the DNA in purified form are well known to one skilled in the art.

The concentration of DNA present in the mixture may be determined empirically for each use, but will generally be in the range of from about 0.001 μg/μl to about 0.1 μg/μl in the slide based embodiment.

Similarly, the concentration of the adherence-promoting polymer can be determined empirically for each use, but will generally be in the range of 0.001% to about 1%.

Similarly, the concentration of the non-proteinaceous, polycationic-based transfection reagent can be determined empirically for each use, but will generally be in the range of 0.05 μM/μl to about 0.1 μM/μl.

Similarly, the concentration of the non-proteinaceous, lipid-based transfection reagent can be determined empirically for each use, but will generally be in the range of 0.025 μg/μl to about 0.1 μg/μl.

The preferred non-proteinaceous, polycationic-based transfection agent for use in the preferred embodiment of the invention is characterized as being able to transfect “less” transfectable cells, such as neuroblastoma, epithelium, CHO, HepC2, or BNL cells and even some primary cells, by standard conventional transfection methods.

The vector containing the appropriate heterologous nucleic acid can be introduced into an appropriate host cell for propagation or expression using well-known techniques. The heterologous nucleic acid sequences can be present as part of a cloning vehicle, e.g., large vector, such as an expression vector (e.g., a plasmid or viral-based vector), or any other transfection vehicle so long as it is capable of transfecting a host cell. Any cloning vehicle may be used in the practice of the invention provided that it will accept a DNA fragment of the desired size. The choice of a suitable vector is dictated, inter alia, by the overall size of the heterologous polynucleotide sequence and the host cell to be employed in the methods of the invention. Thus, for example, when seeking to express a large nucleic acid molecule, it is preferable to use cosmids, phagemids, YACs, and BACs because of their ability to stably propagate large nucleic acid fragments.

If expression of the DNA fragment is desired, the cloning vehicle should further comprise transcription and translation signals next to the site of insertion of the DNA fragment to allow expression of the DNA fragment in the host cell. Preferred vehicles include the pUC series and the pBR series of plasmids.

The heterologous nucleic acid molecules can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion are well known to those of ordinary skill in the art. Procedures for ligating the heterologous nucleic acid, the promoter, terminator and other elements, respectively, and to insert them into suitable cloning vehicles containing the information necessary for replication, are well known to persons skilled in the art (vide e.g., Sambrook et al., 1989; inter alia). Similar procedures, or modifications thereof, can be readily employed to prepare matrices comprising expression vectors in accord with the subject invention.

It may be desirable to express the gene product of interest as a fusion protein. Accordingly, fusion vectors that allow for the production of such peptides are also encompassed. Fusion vectors can increase the expression of a recombinant protein, allow for the visualization of the expressed protein, increase the solubility of the recombinant protein, and aid in the purification of the protein by acting for example as a ligand for affinity purification. A proteolytic cleavage site may be introduced at the junction of the fusion moiety so that the desired peptide can ultimately be separated from the fusion moiety. Proteolytic enzymes include, but are not limited to, factor Xa, thrombin, and enterokinase. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al, Gene 69:301-315 (1988)) and pET 11d (Studier et al., Gene Expression Technology: Methods in Enzymology 185:60-89 (1990)).

As used herein, the term “vector” means an expression construct, e.g., nucleic acid construct wherein a DNA of interest operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. In the present specification, “plasmid” and “vector” are sometimes used interchangeably, as the plasmid is the most commonly used form of vector at present. However, the invention is intended to include such other forms of expression vectors which serve equivalent functions and which become known in the art subsequently hereto.

“Expression vector” includes vectors which are capable of expressing DNA sequences contained therein, where such sequences are operably linked to other sequences capable of effecting their expression. It is implied, although not always explicitly stated, that these expression vectors must be replicable in the host organisms either as episomes or as an integral part of the chromosomal DNA. Clearly a lack of replicability would render them effectively inoperable. In sum, “expression vector” is given a functional definition, and any DNA sequence which is capable of effecting expression of a specified DNA code disposed therein is included in this term as it is applied to the specified sequence. In general, expression vectors of utility in recombinant DNA techniques are often in the form of “plasmids” which refer to circular double stranded DNA loops that, in their vector form are not bound to the chromosome.

“Expression vectors” suitable for use in the practice of the present invention are well known to those of skill in the art and include those that are replicable in eukaryotic cells and/or prokaryotic cells as well as those that remain episomal and those that integrate into the host cell genome. Construction of bacterial or mammalian expression vectors is well known in the art, once the nucleic acid of interest/heterologous nucleic acid is available. For example, DeBoer (U.S. Pat. No. 4,511,433) has disclosed promoters for use in bacterial expression vectors. Sproat et al. Nucleic Acids Res. 13:2959 (1985) and Mullenbach et al. J. Biol. Chem. 261:719 (1986) disclose how to construct synthetic genes for expression in bacteria.

As used herein, the term “expression” refers to any number of steps comprising the process by which nucleic acid molecules are transcribed into RNA, and (optionally) translated into peptides, polypeptides, or proteins. If the polynucleic acid is derived from genomic DNA, expression may, if an appropriate eukaryotic host cell or organism is selected, include splicing of the RNA.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

Generally, recombinant expression vectors will include origins of replication and selectable markers permitting transfection of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), .alpha.-factor, acid phosphatase, or heat shock proteins, among others. The heterologous structural sequence is assembled in appropriate phase with translation initiation and termination sequences. Optionally, the heterologous sequence can encode a fusion protein including an N-terminal identification peptide imparting desired characteristics, e.g., stabilization or simplified purification of expressed recombinant product.

As noted, supra, the expression of the heterologous nucleic acids in mammalian cells is preferred. Thus, for expression in mammalian cells, mammalian expression vectors will be required. Examples of mammalian expression vectors include pCDM8 (Seed, B. Nature 329:840(1987)) and pMT2PC (Kaufman et al., EMBO J 6:187-195 (1987)). Other preferred mammalian expression vectors that contain both prokaryotic sequences, to facilitate the propagation of the vector in bacteria, and one or more eukaryotic transcription units for expressing the target sequence in eukaryotic host cells. Exemplary vectors that can be readily adapted for use in the subject method include the pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors. Some of these vectors may be modified with sequences from bacterial plasmids, such as pBR322, to facilitate replication and drug resistance selection in both prokaryotic and eukaryotic cells.

On the other hand, derivatives of viruses, such as the bovine papillomavirus (BPV-1), or Epstein-Barr virus (pHEBo, pREP-derived and p205) and the like, may also find use in the claimed method(s) of the invention. The various methods employed in the preparation of the plasmids are well known in the art.

Particularly preferred vectors contain regulatory elements that can be linked to the target sequence for transfection of mammalian cells, and include cytomegalovirus (CMV) promoter-based vectors such as pcDNA1 (Invitrogen, San Diego, Calif.), MMTV promoter-based vectors such as pMAMNeo (Clontech, Palo Alto, Calif.) and pMSG (Pharmacia, Piscataway, N.J.), and SV40 promoter-based vectors such as pSVO (Clontech, Palo Alto, Calif.).

The cloning vehicle may also include a selectable marker, such as a gene product, which complements a defect in the host cell, or one that confers antibiotic resistance. Examples of antibiotics useful as Aspergillus selection markers include hygromycin, phleomycin and basta. Other examples of Aspergillus selection markers include amdS, which encodes an enzyme involved in acetamide utilization; pyrG, which encodes an enzyme involved in uridine biosynthesis; argB, which encodes an enzyme involved in arginine biosynthesis; niaD, which encodes an enzyme involved in the nitrate assimilation pathway; and sC, which encodes an enzyme involved in the sulfate assimilation pathway. Preferred for use in an Aspergillus host cell are the amdS and pyrG markers of Aspergillus nidulans or Aspergillus oryzae.

Improvements in DNA vectors have also been made and are likely applicable to all of the non-viral delivery systems. These include the use of supercoiled minicircles reported by RPR Gencell (which do not have bacterial origins of replication nor antibiotic resistance genes and thus are potentially safer as they exhibit a high level of biological containment), episomal expression vectors as developed by Copernicus Gene Systems Inc (replicating episomal expression systems where the plasmid amplifies within the nucleus but outside the chromosome and thus avoids genome integration events) and T7 systems as developed by Progenitor (a strictly a cytoplasmic expression vector in which the vector itself expresses phage T7 RNA polymerase and the therapeutic gene is driven from a second T7 promoter, using the polymerase generated by the first promoter). Other, more general improvements to DNA vector technology include use of cis-acting elements to effect high levels of expression (Vical), sequences derived from alphoid repeat DNA to supply once-per-cell-cycle replication and nuclear targeting sequences (from EBNA-1 gene (Calos at Stanford, with Megabios); SV40 early promoter/enhancer or peptide sequences attached to the DNA).

It is noteworthy that transcription of the heterologous nucleic acid encoding the target protein or gene product of interest by higher eukaryotes can be increased by inserting an enhancer sequence into the vector. Enhancers are cis-acting elements of DNA, usually about from 10 to 300 bp that act on a promoter to increase its transcription. Examples including the SV40 enhancer on the late side of the replication origin (bp 100 to 270), a cytomegalovirus early promoter enhancer, the polyoma enhancer on the late side of the replication origin, and adenovirus enhancers.

Cells suitable for use in the methods of the invention include prokaryotes, yeast, or higher eukaryotic cells, including plant and animal cells, especially mammalian cells. Prokaryotes include gram negative or gram positive organisms. Mammalian cells are preferred, especially primate cells derived from Homo sapiens although other species of mammalian cells may also be used, e.g., feline, canine, bovine, porcine, mouse and rat. The cells can be fully differentiated cells or progenitor/stem cells. The host cells can be derived from normal or diseased tissue, from differentiated or undifferentiated cells, from embryonic or adult tissue.

The cells may be dispersed in culture, or can be tissues samples containing multiple cells which retain some of the microarchitecture of the organ.

The heterologous nucleic acids can also be expressed in insect cells using, for example, baculovirus expression vectors. Baculovirus vectors available for expression of proteins in cultured insect cells (e.g., Sf9 cells) include the pAc series (Smith et al., Mol. Cell Biol. 3:2156-2165 (1983)) and the pVL series (Lucklow et al., Virology 170:31-39 (1989)).

Yeast cells, if used as the transfectable host cells, may be of any species, which are cultivable, and in the gene expression matrix can be maintained upon transfection. Suitable species include Kluyverei lactis, Schizosaccharomyces pombe, and Ustilaqo maydis; Saccharomyces cerevisiae is preferred. Other yeast that can be used in practicing the present invention are Neurospora crassa, Aspergillus niger, Aspergillus nidulans, Pichia pastoris, Candida tropicalis, and Hansenula polymorpha. The term “yeast”, as used herein, includes not only yeast in a strictly taxonornic sense, i.e., unicellular organisms, but also yeast-like multicellular fungi or filamentous fungi.

Exemplary yeast cells include Saccharomyces cerevisiae, or common baker's yeast, which is the most commonly used among eukaryotic microorganisms. Other strains are available and may be substituted for the baker's yeast.

The heterologous nucleic acids can also be expressed by expression vectors that are operative in yeast. For expression in Saccharomyces, the plasmid YRp7, for example, (Stinchcomb, et al, Nature, 282:39 (1979); Kingsman et al, Gene, 7:141 (1979); Tschemper, et al, Gene, 10:157 (1980)) is preferred. Other representative promoters that are functional in yeast and required for the expression of the heterologous nucleic acid in yeast include the promoters for metallothionein, 3-phosphoglycerate kinase (Hitzeman et al., J. Biol. Chem. 255, 2073 (1980) or other glycolytic enzymes (Hess et al., J. Adv. Enzyme Req. 7, 149 (1968); and Holland et al. Biochemistry 17, 4900 (1978)), such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phospho-glucose isomerase, and glucokinase.

Where yeast is the host cell, expression of the heterologous nucleic acid may be facilitated by expression vectors that are operative in yeast. Examples of vectors for expression in yeast e.g., S. cerevisiae include pYepSec1 (Baldari, et al., EMBO J. 6:229-234 (1987)), pNFa (Kuijan et al., Cell 30:933-943(1982)), pJRY88 (Schultz et al., Gene 54:113-123 (1987)), and pYES2 (Invitrogen Corporation, San Diego, Calif.). A number of other vectors are known that are capable of expressing recombinant proteins in yeast. Representative examples include YEP24, YIP5, YEP51, YEP52, and YRP17, all of which are suitable cloning and expression vehicles useful in the introduction of genetic constructs into S. cerevisiae. See Broach et al. (1983) in Experimental Manipulation of Gene Expression, ed. M. Inouye Academic Press, p. 83, incorporated by reference herein).

The present invention also encompasses a nucleic acid containing matrix comprising a surface having affixed thereto, in indexed locations, nucleic acid of known sequence or source by a method described herein. Unknown DNA's can also be used.

Likewise, transfected cells—cells which have been transfected with a heterologous nucleic acid in accordance with any of the herein disclosed methods are also envisioned by the present invention as are methods of introducing a heterologous nucleic acid molecule into a host cell in accordance with the method of the invention.

The invention further includes identification or detection of host cells transfected with a heterologous nucleic acids, i.e., cDNA, which is introduced into the cells.

In accordance with the above, the presence of the heterologous DNA may be determined by introducing a labeled-oligonucleotide probe that is sufficiently complimentary to a portion of the nucleotide sequence of the heterologous DNA such as to form a complex (hybrid) with the portion of the heterologous DNA sequence, at moderately stringent condition. Identification of transfected or transfected cells is achieved via identification of the hybrid/complex.

Methods for making a gene expression matrix for use in the methods of the invention are also provided. The method dictates affixing nucleic acid molecules to a surface such that when cells are plated onto the surface bearing the nucleic acid, the nucleic acid is capable of being introduced into the cells (i.e., the surface facilitates the spatial adherence of the nucleic acid until the transfer or movement of the nucleic acid from the surface into the cells).

Any suitable surface that can be used to affix the nucleic acid containing mixture to its surface can be used. For example, the surface can be glass, plastics (such as polytetrafluoroethylene, polyvinylidenedifluoride, polystyrene, polycarbonate, polypropylene), silicon, metal, (such as gold), membranes (such as nitrocellulose, methylcellulose, PTFE or cellulose), paper, biomaterials (such as protein, gelatin, agar), tissues (such as skin, endothelial tissue, bone, cartilage), minerals (such as hydroxylapatite, graphite). Any surface that has partitions can be condsidered a plate for use in this invention.

In certain embodiments, the surface of the matrix can be coated with detectable molecules that impart functionality to the matrix, e.g., add detection properties to the overall matrix. Representative examples include antibodies, biotin, avidin, Ni-NTA to bind epitopes, avidin, biotinylted molecules, or 6-His tagged molecules. Alternatively, the molecules that aid in the detection of a complex between the transfected cells and their respective binding partners can be part of the culture reagents.

It is noteworthy that the surface of the matrix need not be a flat surface per se. Microsphere (bead) may also suffice as a surface onto which a plurality of nucleic acids are deposited in indexed locations in accordance with a method of the invention. To be most effective, it is preferable that each bead be distinct in that it comprise an individual feature, e.g., have a homogenous population of nucleotide acid sequences. Tags and methods of applying the same to any given bead are well known and will provide an effective means of identifying one bead from another in a mixture of beads, thereby allowing one to identify the nucleic acid that is displayed on a particular bead via conventional techniques.

In an alternative format, the gene expression matrix of the invention may be engineered to provide a plurality of different heterologous nucleic acids in a given area. The matrix may comprise various areas (“feature”) having deposited thereon nucleic acids encoding different products. “Feature” as used herein to describe a matrix of the invention refers to an area of a matrix (substrate) having a homogeneous collection of a target heterologous nucleotide sequence. Thus, one feature differs from another if the target sequence differs in their nucleotide sequence. This aspect of the invention will allow one skilled in the art to effectively co-transfect host cells with two different nucleotide sequences. Thus, as used herein, co-transfection refers to the transfection of a host cell with at least two or more heterologous nucleic acid constructs, which will effectively allow the cell to express at least two gene products.

In an exemplary method, one of the two plasmids used in the co-transfection may encode a marker protein, thereby allowing one skilled in the art to infer expression of a particular protein based upon the detection of the marker protein. Other uses of a gene expression matrix that allows for the co-transfection of a host cell will be readily apparent to one skilled in the art. Methods of co-transfection are well known and readily apparent to one skilled in the art.

Potential Uses

As the study of signal transduction, or the flow of information throughout the cell, has broadened and matured, it has become apparent that many medically significant biological processes are mediated by proteins participating in signal transduction pathways that involve G-proteins and/or second messengers, e.g., cAMP (Lefkowitz, Nature, 351:353-354 (1991)). Some examples of these proteins include the GPC receptors, G-proteins themselves, effector proteins, e.g., phospholipase C, adenyl cyclase, ion channels, and phosphodiesterase, and actuator proteins, e.g., protein kinase A and protein kinase C (Simon, M. I., et al., Science, 252:802-8 (1991)).

Since the activities of a G protein and its effector molecules are amenable to assay, assays focused on any of these molecules may be used to identify potential binding partners for various cell surface receptors and modulators thereof.

The activity of a G-protein coupled receptor may be measured using any of a variety of functional assays which are well-known in the art, in which activation of the receptor in question results in an observable change in the level of some second messenger, including but not limited to adenylate cyclase, calcium mobilization, arachidonic acid release, ion channel activity, inositol phospholipid hydrolysis or guanylyl cyclase.

Cells expressing the receptor in question are transfected in accordance with a method of the invention and are used to obtain the desired second messenger coupling. Receptor activity may also be assayed in an oocyte expression system, using methods well known in the art.

Binding partners that target G-protein coupled receptors expressed by cells following transfection by any of the herein disclosed methods, but do not elicit a second messenger response such that the activity of the G-protein coupled receptors is prevented is also contemplated by the present invention.

“Signal transduction” is the processing of physical or chemical signals from the cellular environment through the cell membrane, and may occur through one or more of several mechanisms, such as activation/inactivation of enzymes (such as proteases, or other enzymes which may alter phosphorylation patterns or other post-translational modifications), activation of ion channels or intracellular ion stores, effector enzyme activation via guanine nucleotide binding protein intermediates, formation of inositol phosphate, activation or inactivation of adenylyl cyclase, direct activation (or inhibition) of a transcriptional factor.

(i) Assays:

A variety of methods exist which can be used to detect the effects on a transfected host cell by the uptake of the heterologous nucleic acid.

A convenient assay to detect changes on a host cell conferred by the uptake of a heterologous DNA is to detect changes in cell morphology. In general, such an assay measures changes in cell structure, which are ultimately detected by conventional means known to one skilled in the art. Exemplary methods useful for measuring morphological changes include, but are not limited to differential sedimentation, differential buoyant density, atomic force microscopy, electron microscopy etc.

Alternatively, the phenotypic changes attending host cell transfected with a heterologous nucleic acid may be assessed by detecting the level of a particular protein or level of mRNA corresponding to the protein in question.

Also a part of the invention are methods for identifying modulators, i.e., candidate or test compounds or agents (e.g., peptides, peptidomimetics, small molecules or other drugs) which bind to target proteins or polypeptides or biologically active fragments thereof, have a stimulatory or inhibitory effect on, for example, cell surface expression of a receptor protein or its activity, or have a stimulatory or inhibitory effect on, for example, the expression or activity of a receptor specific substrate.

Synthetic compounds, natural products, and other sources of potentially biologically active materials can be screened in any of the herein disclosed assays.

In the assays discussed infra, the procedures involves providing appropriate cells which express the target receptor polypeptide, following transfection in accordance with an embodiment of the invention. Such cells include cells from mammals, yeast, Drosophila or E. coli. Mammalian cells are preferred. Essentially, cells expressing the target receptor are contacted with a test compound to observe binding, stimulation or inhibition of a functional response.

Binding partners for a target molecule of interest can be identified by engineering a gene expression matrix that expresses a potential target and contacting the matrix with appropriate amounts of the target molecule to see if a binding event occurs. The binding can be identified by conventional techniques, some of which are detailed herein.

For example, immunofluorescence may be used to detect expression of a gene product of interest, e.g., the gene product encoded by the target heterologous DNA. The method proposes using an antibody that binds the protein encoded by the heterologous DNA and is fluorescently labeled. The antibody or other suitable binding partner may be added to the matrix under conditions favoring a binding event between the antibody and the protein and the location, e.g., area of the surface containing the protein can be identified by detecting fluorescence. The presence of fluorescence generally suggests that the gene product encoded by the heterologous DNA has been expressed in the defined location(s) which show fluorescence.

In an alternative embodiment, a matrix of transfected cells, following transfection in accordance with a method of the invention, is incubated with labeled cognate target, and an unlabeled target compound and the binding event between the unlabeled target compound and the surface of the matrix bearing the transfected cells is determined by measuring a decrease in the signal of the label due to competition between the cognate labeled target and the unlabeled target compound for the transfected cells. Preferably, the labeled cognate target is incubated with the matrix before incubation with the unlabeled target.

In the case where the encoded protein confers a change in the cell signal transduction pathways, the phenotypic change may be assessed via measurement of second messenger activity etc. Some exemplary assays are detailed herein.

In some instances, the gene expression matrix of the invention can be engineered to provide a variegated library of expression vectors that can ultimately be used to assess the ability of one or more of its members to induce or inhibit signal transduction.

An embodiment of the invention provides a process for determining whether a test compound specifically binds to and activates a target receptor protein, which comprises contacting host cells expressing on their cell surface the target protein, following transfection with any of the herein disclosed methods of the invention with the test compound under conditions suitable for activation of the target protein, and measuring the second messenger response in the presence and in the absence of the test compound, a change in second messenger response in the presence of the test compound indicating that the test compound activates the target receptor protein.

In one such screen, the procedure involves use of mammalian cells (CHO, HEK293, Xenopus Oocytes, etc.) which are transfected to express the receptor of interest, and which are also transfected with a reporter gene construct that is coupled to activation of the receptor (for example, luciferase or beta-galactosidase behind an appropriate promoter). The cells are contacted with a test substance and the receptor agonist (ligand) and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

In another screening procedure, the method involves the use of mammalian cells (CHO, HEK 293, Xenopus Oocytes, etc.) which are transfected to express the receptor of interest, by any of the methods of the invention. The cells are loaded with an indicator dye that produces a fluorescent signal when bound to calcium, and the cells are contacted with a test substance and a receptor agonist. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

Another screening technique involves expressing a target receptor protein on a recombinant host cell in accordance with a method of the invention in which the receptor is linked to phospholipase C or D. Representative examples of such cells include, but are not limited to, endothelial cells, smooth muscle cells, and embryonic kidney cells. The screening may be accomplished as described herein by detecting activation of the receptor or inhibition of activation of the receptor from the phospholipase second signal.

Another screening technique for antagonists or agonists involves introducing RNA encoding the target polypeptide into Xenopus oocytes (or CHO, HEK 293, etc.) to transiently or stably express the receptor. Following transfection in accordance with a method of the invention, the receptor oocytes are then contacted with the receptor ligand and a compound to be screened Inhibition or activation of the receptor is then determined by detection of a signal, such as, cAMP, calcium, proton, or other ions.

Another method involves screening for target receptor polypeptide inhibitors by determining inhibition or stimulation of the target receptor polypeptide-mediated cAMP and/or adenylate cyclase accumulation or diminution. Such a method involves transiently or stably transfecting a eukaryotic cell with a heterologous DNA encoding the target polypeptide receptor under conditions favoring expressing of the target receptor on the cell surface. The cell is then exposed to potential antagonists in the presence of target receptor polypeptide ligand. The changes in levels of cAMP is then measured over a defined period of time, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist binds the receptor, and thus inhibits the target receptor polypeptide-ligand binding, the levels of the target receptor polypeptide-mediated cAMP, or adenylate cyclase activity, will be reduced or increased.

When using yeast cells as host cells, the gene expression matrix can be engineered to transfect these cells to express and secrete small peptides, some of which can permit autocrine activation of heterologously expressed human (or mammalian) G-protein coupled receptors (Broach, et al., Nature 384: 14-16, 1996; Manfredi, et al., Mol. Cell. Biol. 16:4700-4709, 1996). This provides a rapid direct growth selection (e.g., using the FUS1-HIS3 reporter) for surrogate peptide agonists that activate characterized or orphan receptors.

Alternatively, yeast cells that functionally express human (or mammalian) G-protein coupled receptors linked to a reporter gene readout (e.g., FUS1-LacZ) can be used as a platform for high-throughput screening of known ligands, fractions of biological extracts and libraries of chemical compounds for either natural or surrogate ligands. Functional agonists of sufficient potency (whether natural or surrogate) can be used as screening tools in yeast cell-based assays for identifying G-protein coupled receptor antagonists. For example, agonists will promote growth of a host cell with FUS-HIS3 reporter or give positive readout for a host cell with FUS1-LacZ. However, a candidate compound that inhibits growth or negates the positive readout induced by an agonist is an antagonist. For this purpose, the yeast system offers advantages over mammalian expression systems due to its ease of utility and null receptor background (lack of endogenous G-protein coupled receptors) which often interferes with the ability to identify agonists or antagonists.

Another method involves screening for compounds, which are antagonists, and thus inhibit activation of the target receptor polypeptide by determining inhibition of binding of labeled ligand to cells that have the receptor on the surface thereof, or cell membranes containing the receptor. Such a method involves transfecting a eukaryotic cell with a heterologous DNA encoding the target polypeptide such that the cell expresses the receptor on its surface in accordance with a method of the invention. The transfected host cell is then contacted with a potential antagonist in the presence of a labeled form of a ligand. The ligand can be labeled, e.g., by radioactivity. The amount of labeled ligand bound to the receptors is measured, e.g., by measuring radioactivity associated with transfected cells or membrane from these cells. If the compound binds to the target receptor polypeptide, the binding of labeled ligand to the target receptor polypeptide is inhibited as determined by a reduction of labeled ligand, which binds to the target receptors. This method is called a binding assay. Naturally, this same technique can be used to look for an agonist.

It is also within the scope of this invention to determine the ability of a compound (e.g., a substrate of the target protein) to interact with its binding partner (cells expressing a cell surface receptor without the labeling of any of the interactants. For example, a microphysiometer can be used to detect the interaction of a compound with the cell surface receptor protein without the labeling of either the compound or the cells expressing the mammalian cell surface receptor. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between the receptor protein and its substrate.

In certain embodiments, the gene expression matrix can be engineered to express a library of related, mutated DNA sequences e.g., a library of mutants of a particular target protein, in which the coding sequence differs from a naturally occurring sequence by deletion, substitution or addition of at least one residue and the binding affinity of the mutants assessed using conventional techniques. The prior art is replete with numerous mutagenesis protocols, any one of which may be utilized to provide the library of mutant nucleic acids. See Ruf et al. (1994) Biochemistry 33:15 65-1572; Wang et al. (1994) J. Biol. Chem. 269:3095-3099; Gustin et al. (1993) Virology 193:653-660; Brown et al. (1992) Mol. Cell Biol. 12:2644-2652; Meyers et al. (1986) Science 232:613); Leung et al. (1989) Method Cell Mol Biol 1:11-19); Miller et al. (1992) A Short Course in Bacterial Genetics, CSHL Press, Cold Spring Harbor, N.Y.; and Greener et al. (1994) Strategies in Mol Biol 7:32-34).

As an example, the mutant protein can be assayed for the ability to (1) interact with (e.g., bind to) a potential target or binding partner; (2) regulate the phosphorylation state of a ion channel protein or portion thereof; (3) associate with (e.g., bind) a particular ion e.g., calcium and, for example, act as a calcium dependent kinase, (4) modulate an ion channel mediated activity in a cell to, for example, beneficially affect the cell; (5) modulate the release of second messengers; and where appropriate (6) modulate membrane excitability; (7) influence the resting potential of membranes; (8) modulate wave forms and frequencies of action potentials; and (9) modulate thresholds of excitation.

Representative but non-limiting examples are provided herein wherein the recombinant cells are transfected with a heterologous nucleic acid encoding a chemokine receptor in accordance with a method of the invention and wherein the cells express on their surface the heterologous chemokine receptor.

(i) Direct Assay to Determine Receptor/Ligand Binding

Detectably labeled test compounds are exposed to membrane preparations presenting target chemokine receptors in a functional conformation. For instance, HEK293 cells, or other tissue culture cells, are transfected with an expression vehicle encoding a chemokine receptor in accordance with a method of the invention. A membrane preparation is then made from the transfected cells expressing the chemokine receptor. The membrane preparation is exposed to a radio-labeled test compounds (e.g., chemokines) and incubated under suitable conditions (e.g., 10 minutes at 37° C.). The membranes, with any bound test compounds, are then collected on a filter by vacuum filtration and washed to remove unbound test compounds. The radioactivity associated with the bound test compound is then quantitated by subjecting the filters to liquid scintillation spectrophotometry. The specificity of test compound binding may be confirmed by repeating the assay in the presence of increasing quantities of unlabeled test compound and noting the level of competition for binding to the receptor.

These binding assays can also identify modulators of chemokine receptor binding. The previously described binding assay may be performed with the following modifications. Modulators of chemokine receptor function may be identified using a similar assay. The membrane preparation displaying a chemokine receptor is exposed to a constant and known quantity of a functional ligand. In addition, the membrane-bound chemokine receptor is also exposed to an increasing quantity of a test compound suspected of modulating the activity of that chemokine receptor. If the levels of filter-associated label correlate with the quantity of test compound, that compound is a modulator of the activity of the chemokine receptor. If the level of filter-associated label increases with increasing quantities of the test compound, an activator or potentiator has been identified. In contrast, if the level of filter-associated label varies inversely with the quantity of test compound, an inhibitor of chemokine receptor activity has been identified. Confirmation of the identification of a modulator can be achieved by competing a labeled form of the modulator with varying quantities of an unlabeled form of the modulator using the assay described above for confirming ligand identities.

(ii) Assays Measuring Second Messenger Activity

Phospholipase C Activity—A suitable assay for ligands or modulators involves monitoring phospholipase C activity, as described in Hung et al., J. Biol. Chem. 116:827-832 (1992). Initially, recombinant host cells, transfected in accordance with a method of the invention with a heterologous nucleic acid encoding a chemokine receptor and expressing on a surface thereof the chemokine receptor are loaded with ^{.3}H-inositol for about 24 hours. Test compounds (i.e., potential ligands) are then added to the transfected cells, which may still be bound to a matrix of the invention and incubated at 37° C. for about 15-45 minutes, preferably 15 minutes. Thereafter, the cells are then exposed to a solubilizing solution e.g., 20 mM formic acid to solubilize and extract hydrolyzed metabolites of phosphoinositide metabolism (i.e., the products of phospholipase C-mediated hydrolysis). The extract is subjected to anion exchange chromatography using a conventional column, e.g., an AG1X8 anion exchange column (formate form). Inositol phosphates are eluted with 2 M ammonium formate/0.1 M formic acid and the ³H associated with the compounds is determined using liquid scintillation spectrophotometry.

The phospholipase C assay can also be exploited to identify modulators of chemokine receptor activity. The aforementioned assay is performed as described above, but with the addition of a potential modulator. Elevated levels of detectable label would indicate the modulator is an activator, depressed levels of the label would indicate the modulator is an inhibitor of chemokine receptor activity. Other variations of the phospholipase C assay are well known and incorporated herein in their entirety.

(iii) GTP Hydrolysis

The association of chemokine receptors with G proteins affords the opportunity of assessing receptor activity by monitoring G protein activities. For example, a characteristic activity of G proteins, GTP hydrolysis, may be monitored using, for example, ³²P-labeled GTP.

Thus, an embodiment of the invention comprehends indirect assays for identifying receptor ligands that exploit the coupling of chemokine receptors to G proteins. As reviewed in Linder et al., Sci. Am., 267:56-65 (1992), during signal transduction, an activated receptor interacts with a G protein, in turn activating the G protein. The G protein is activated by exchanging GDP for GTP. Subsequent hydrolysis of the G protein-bound GTP deactivates the G protein. Chemokine receptor activation is often associated with intracellular calcium ²⁺flux. See also Signal Transduction: A Practical Approach. G. Milligan, Ed. Oxford University Press, Oxford England.

Importantly, parallel assays, using either technique, may be performed in the presence and absence of putative ligands. This type of GTP hydrolysis assay is also useful for the identification of modulators of chemokine receptor binding.

Other methods to detect ligand/receptor mediated interactions may also be used, e.g. a cAMP assay. Cyclic adenosine monophosphate (cAMP) is a second messenger that mediates the biologic responses of cells to a wide range of extracellular stimuli. Upon stimulation, such as by the binding of an appropriate agonist to specific cell surface receptors, adenylate cyclase is activated to convert adenosine triphosphate (ATP) to cAMP. As such, ligand binding to a target receptor e.g., a chemokine receptor that activates a G protein, which in turn activates adenylyl cyclase, can be detected by monitoring cAMP levels.

Other screening techniques include the use of cells, which express the target polypeptide following transfection with any of the methods of the invention in a system which measures extracellular pH changes caused by receptor activation. In this technique, compounds may be contacted with cells expressing the receptor polypeptide. A second messenger response, e.g., signal transduction or pH changes, is then measured to determine whether the potential compound activates or inhibits the receptor.

(iv) Dye

Chemokine receptor activation often is associated with an intracellular Ca⁺⁺ flux. Thus, recombinant cells expressing a heterologous DNA encoding a chemokine receptors may be loaded with a calcium-sensitive dye. Upon activation of the expressed receptor, a Ca⁺⁺ flux would be rendered spectrophotometrically detectable by the dye. Alternatively, the Ca⁺⁺ flux could be detected microscopically.

ii. Therapeutic

The present invention also pertains to uses of novel compounds identified by any of the assays described herein for diagnoses, prognoses, and treatments of various disease states.

In an embodiment of the invention, a gene expression matrix may be used to further characterize compound libraries generated as above. For example, a library of compounds previously identified may be further characterized in repeated processes to identify compounds having a desired characteristics, e.g. high binding affinity, ability to antagonize a G protein coupled receptor etc. According to this method, previously identified compounds may be exposed to a gene expression matrix that has been engineered to express not only the target protein initially identified in a previous screen to bind a particular compound but also various mutants thereof in an effort to identify those compounds having the desired characteristics, e.g., binding affinity. The process may be repeated until a compound with the desired characteristics is identified.

Accordingly, it is within the scope of the present invention to use such compounds in the design, formulation, synthesis, manufacture, and/or production of a drug or pharmaceutical composition for use in diagnosis, prognosis, or treatment of diseases that would benefit from the compounds identified herein. Furthermore, this invention pertains to uses of novel agents identified by the above-described screening assays for treatments as described herein.

iii. Antisense Constructs and Uses Thereof

In addition to the above, another aspect of the invention pertains to the introduction of antisense constructs prepared through the use of antisense technology, which may be used to control gene expression through triple-helix formation or antisense DNA or RNA, both of which methods are based on binding of a polynucleotide to DNA or RNA.

Consequently, antisense-constructs—DNA or RNA that are taken up by suitable host cells but are not expressed and instead block the expression of certain genes in vivo are also contemplated by the methods of the invention.

The prior art is replete with teachings showing that short antisense oligonucleotides can be imported into cells where they act as inhibitors, despite their low intracellular concentrations caused by their restricted uptake by the cell membrane. (Zamecnik et al., Proc. Natl. Acad. Sci. USA 83, 4143-4146 [1986]). The oligonucleotides can be modified to enhance the uptake, e.g., by substituting their negatively charged phosphodiester groups by uncharged groups.

For the purpose of this invention, the intended objective with respect to DNA of the cell is to interfere with its replication and transcription. Likewise, with respect to RNA, it is an object of the invention to interfere with all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of the proposed effect within the context of the above embodiment—is the modulate of the expression of the target nucleic acid in the host cell. As used within the context of this embodiment, a “target nucleic acid” is intended to cover DNA or RNA whose replication or transcription is intended to be inhibited. As such, “modulation” with respect to the above referenced embodiment means a decrease or complete inhibition in the expression of the “target nucleic acid”.

The technique for the above embodiment proposes engineering an oligonucleotide so as to be complementary to a region of the gene involved in transcription of the mRNA to be targeted. The antisense RNA oligonucleotides is thereafter introducing into suspected host cells in accordance with a method of the invention and under conditions favoring its uptake and subsequent expression whereby it hybridizes to mRNA in vivo and blocks translation of the mRNA molecules into proteins (triple helix—see Lee et al., Nucl. Acids Res., 6:3073 (1979); Cooney et al, Science, 241:456 (1988); and Oligodeoxynucleotides as Antisense Inhibitors of Gene Expression, CRC Press, Boca Raton, Fla. (1988).

For practicing the above embodiments, the antisense DNA sequences may use natural nucleotides or unnatural nucleotide mimics known in the art.

Alternatively, the gene expression matrix can be engineered to express a double stranded RNA molecule (dsRNA) and its effect e.g., corresponding endogenous RNA in the transfected host cells assessed via conventional means. Techniques for introducing dsRNA are known to one skilled in the art. See Fire A (1999) Trends Genet. 15:358-363; Sharp P A (1999) Genes Dev 13:139-141; and Hunter C (1999) Curr. Biol. 9:R440-R442. Thus, the dsRNA approach described above (e.g., RNAi) will prove effective in inactivating a cloned gene as well as in establishing gene expression profiling etc. See Fire et al. (1998) Nature 391: 80681 1; and Montgomery et al. (1998) PNAS 95:15502-15507). In an alternative embodiment, method(s) of the invention may be used to introduce a DNA fragment, which is anti-sense to an mRNA encoding a receptor for a drug. It is believed that the anti-sense heterologous DNA, will effectively decrease the expression of the drug receptor protein, thereby causing a decrease in drug binding to cells containing the anti-sense DNA, thus enabling one to further characterize a potential target etc. Other uses will become apparent to one skilled in the art.

The invention also encompasses vectors in which the heterologous nucleic acids are cloned into the vector in reverse orientation, but operably linked to a regulatory sequence that permits transcription of antisense RNA. Thus, an antisense transcript can be produced to all, or to a portion, of a target nucleic acid including both coding and non-coding regions

In yet another embodiment, the heterologous nucleic acid molecules for use in the methods of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1):5-23).

As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93:14670-675.

Accordingly, PNAs of target nucleic acid molecules can be used in therapeutic and diagnostic applications. For example, PNAs can be used as antisense agents for sequence-specific modulation of gene expression by, for example, inducing transcription or translation arrest or inhibiting replication.

The methods described herein may also be used for the purpose of RNA interference (RNAi) studies. Introduction of small interfering RNA (siRNA) molecules into cell lines or primary cultures using the methods described herein e.g., MAGEC would enable high-throughput evaluation of gene function in living cells. The flexibility of the herein described MAGEC platform enables the use of double-stranded oligonucleotide siRNAs as well as siRNAs expressed from plasmids. In one embodiment, libraries or collections of siRNAs may be rapidly and efficiently delivered to cultured cells using the methods of the present invention. Cells transfected with the desired siRNAs may be subsequently monitored for changes in growth rate or morphology using high-content screening methods or evaluated in a desired functional assay.

iv. Animal Models

It is also within the scope of this invention to further use a compound (agent) identified as described supra, in an appropriate animal model. For example, an agent identified as described herein (e.g., a GPCR modulating agent, an antisense GPCR nucleic acid molecule, a GPCR-specific antibody, or a GPCR-binding partner) can be used in an animal model to determine the efficacy, toxicity, or side effects of treatment with such an compound.

Alternatively, a compound identified as described supra, can be used in an animal model to determine its mechanism of action.

### EXEMPLARY EMBODIMENTS

### Example 1

Plate-based, Polyethylenimine (PEI)/DNA Embodiment

Overview of MAGEC Plate-Based, PEI Transfection Protocol: Assay as Written is for a 24-well Tissue Culture Assay Format)

Overview: The target nucleic acid is mixed with a linear polyethylenimine reagent and an adherence-promoting polymer such as polyvinyl alcohol, glycogen or methylcellulose. The adherence-promoting polymer, as the name implies, promotes adherence of the PEI/nucleic acid complex to the surface of the multi-well plate. The coated plates are incubated overnight at 4° C. and subsequently dried in vacuo. The plates are then seeded with a cell type of interest to initiate transfection.

Detailed Protocol:

1. In a 1.5 ml tube, add 0.5-4 μg DNA to 50 μl of transfection-enhancing buffer (i.e., EC buffer Qiagen) containing 0.3M sucrose and mix.
2. In another 1.5 ml tube, add 13 μl PEI (ExGen 500 MBI Fermentas) to 50 μl of NaCl (150 mM) mix.
3. Add the PEI/NaCl reagent to the nucleic acid containing tube and mix gently.
4. Incubate at room temperature for 10 minutes.
5. Add 110 μl of the desired cell adherence-promoting polymer (PVA, glycogen or methyl cellulose) and remix the solution.
6. Pipet the sample into a well of multi-well plate. The plates can be uncoated or treated with cell adhesion/matrix proteins (i.e, poly-D-lysine, Matrigel, laminin, and collagen).
7. Spread solution to cover the entire bottom of the well and let sit overnight at 4° C.
8. Dry plate with no heat in a SpeedVac or vacuum chamber. Dried plates can be used immediately or stored at 4° C. for an extended period.
9. One day prior to transfection, cells are passaged to maintain growth in log-phase.
10. On the day of transfection, cells are trypsinized and subsequently seeded into the previously coated and dried multi-well plate(s). The culturing conditions will vary depending on cell type but, preferred conditions for EK293T cell are Dulbecco's Modified Eagles Medium (MEM) containing 10% BCS with L-glutamine and penicillin/streptomycin (pen/strep).
11. Approximately forty hours after plating, the cells are analyzed by the desired assay.

### Example 2

Slide-based, Lipid/DNA Embodiment

Overview of MAGEC Slide-based, Lipid Transfection Protocol:

The nucleic acid of interest is mixed with a lipid-based transfection reagent and an adherence-promoting, non-proteinaceous polymer, such as polyvinyl alcohol (PVA) that promotes adherence to the surface of the glass slide. The mixture is subsequently deposited on the slide and allowed to dry. The printed slides are placed in a culture dish and the cell type of interest is plated over the slides to initiate transfection.
1. 0.8-1.6 μg of target DNA is mixed with about 15 μl transfection-enhancing buffer (i.e., EC buffer Qiagen) containing 0.3M sucrose.
2. 1.5 μl enhancer reagent (QIAGEN) is then added and the mixed by pipetting and then allowed to sit at room temperature for 5 minutes.
3. Transfection Reagent: About 5 μl of lipid reagent (Effectene QIAGEN) is added to the mixture obtained in 2) above. The resulting lipid-DNA mixture is then gently vortexed and allowed to incubate for 10 minutes at room temperature.
4. An equal volume of an adherence-promoting polymer (PVA or other polymers) is added to the mixture, which is then mixed by pipetting.
5. The resulting DNA-lipid mixture is then deposited onto positively-charged glass slides (Superfrost Plus, Fisher Scientific) either manually or using a microarrayer.
6. The slides are allowed to air dry and are stored desiccated at about 4° C.
7. About 18-24 hours prior to transfection, suitable host cells e.g., HEK293T, are passaged to maintain growth in log-phase.
8. The cells from 7) are then trypsinized and subsequently seeded into a petri dish containing the previously printed slide(s) and then incubated at the appropriate culturing conditions. The culturing conditions will vary depending on cell type, preferred conditions for HEK293T cell are Dulbecco's Modified Eagles Medium (DMEM) containing 10% BCS with L-glutamine and penicillin/streptomycin (pen/strep).
9. After about 40 hours, the slides are assessed for transfection efficiency and/or analyzed by any of a number of known methods for expression of a relevant protein.

### Example 3

Plate-based, Lipid/DNA Transfection Embodiment:

Overview of MAGEC Plate-based, Lipid Transfection Protocol:

(Assay as Written is for a 24-well Tissue Culture Assay Format)

The nucleic acid of interest is mixed with a lipid-based transfection reagent and deposited into a well of a multi-well plate coated with poly-lysine, laminin, collagen, or Matrigel. The addition of an adherence-promoting polymer is not essential for efficient transfection in the plate assay; however, the addition of adherence reagents (such as PVA, glycogen or methylcellulose) may enhance cell viability and thus improve transfection efficiencies. The nucleic acid/lipid coated plates are incubated overnight at 4° C. and subsequently dried in vacuo. The plates are then seeded with a cell type of interest to initiate transfection.
1. In a 15 ml tube, add 1.60-4 μg of the target DNA to 63 μl of transfection-enhancing buffer (i.e., EC buffer Qiagen) in which sucrose has been dissolved to a concentration of 0.3.
2. Add 5 μl of Enhancer solution, mix the contents by pipetting five times, and incubate the mixture at room temperature for 5 minutes.
3. Add 15 μl Effectene transfection reagent; and gently mix the solution by vortexing.
4. Incubate at room temperature for 10 minutes.
5. Add 90 μl water, or an adherence-promoting polymer (glycogen, PVA, or PVP), adhere), mix the solution by vortexing and pipette 190 μl into a well of multi-well plate. The plates can be uncoated, or treated with cell adhesion/matrix proteins (i.e.: PDL, matrigel, Laminin, collagen).
6. Spread solution to cover the whole bottom of the well and let sit overnight at 4° C.
7. Dry plate without heat, e.g. in SpeedVac or in a vacuum chamber. The dried plate can be stored at 4° C. for an extended period.
8. About 18-24 hours prior to transfection, the host cells such as those exemplified in previous example are passaged to maintain growth in log-phase and split (provide splitting conditions) Is it the same as the reverse-transfection method—see left column page 20.
9. On day of transfection, the host cells are trypsinized and subsequently seeded into previously coated and dried multi-well plates. (Provide culturing conditions)
10. After about approximately 40-48 hours, the slides are assessed for transfection efficiency and/or analyzed by any of a number of known methods for expression of a relevant protein (Calcium-flux assays, PI hydrolysis, binding assays). (Were any such assays performed, if so provide details).

### Example 4

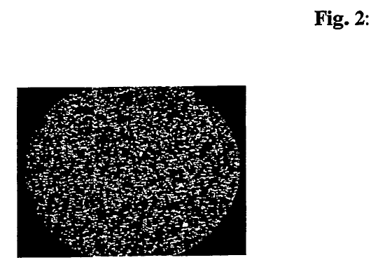

Referring to FIG. 2, shown therein is a scan of emerald green fluorescence protein (EGFP) expression in HEK293 cells. The cells were transfected by the MAGEC plated-based, PEI method according to Example 1. Briefly, the transfected EGFP-expression vector construct was adhered to the surface of a 24-well tissue culture plate with a polycationic transfection reagent (PEI), an adherence-promoting polymer (PVA) and a transfection-enhancing buffer. The resulting mixture was allowed to sit for 16 hours at 4° C. then dried in vacuo. The wells were then plated with HEK293 cells and allowed to culture for 40 hours.

### Example 5

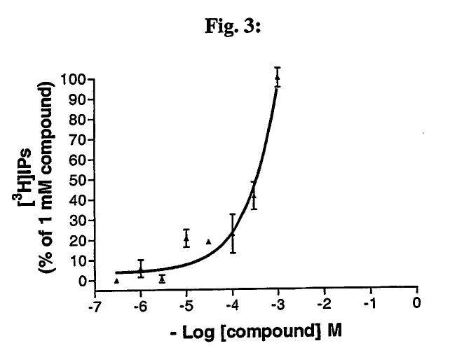

FIG. 3 depicts the effect of varying concentrations of a compound on PI hydrolysis in HEK293 cells transfected by the MAGEC plate-based, PEI method. (Example 1). Briefly, HEK293 cells were co-transfected with two different heterologous nucleic acid molecules, encoding a G protein coupled receptor (human mGluR2) and the promiscous G protein—Galpha16. Co-transfection of cells with heterologous mGluR2 and Galpha16 allows for coupling of the receptor (through activation of endogenous phospholipase C) and therefore permits functional response to agonist (eg. glutamate) to be measured. Glutamate-dependent activation of mGluR2 may thus be determined by a phosphoinositide (PI) hydrolysis assay. The assay measures mGluR2 activation to increasing concentrations of glutamate.

According to the method, the nucleic acids to be introduced were adhered to the surface of a 24-well tissue culture plate containing a polycationic transfection reagent (PEI), an adherence-promoting polymer (PVA) and a transfection-enhancing buffer. The resulting mixture was allowed to sit for 16 hours at 4° C. then dried in vacuo. The wells were then seeded with HEK293 cells and allowed to culture for 24 hours before the start of the assay. The functional assay data points represent mean±SE from three replicates.

### Example 6

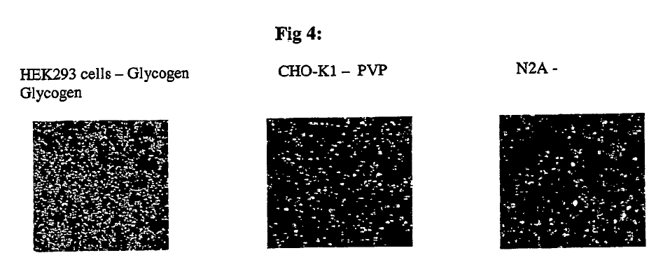

Referring to FIG. 4, shown therein are various images (scans) of different emerald green fluorescence protein (EGFP) expression in HEK293, CHO K1 and N2A cells transfected by the MAGEC plated-based, lipid method—Example 3. The transfected EGFP-expression vector construct was adhered to the surface of a 96-well tissue culture plate with a lipid-based transfection reagent (Effectene), an adherence-promoting polymer as noted and a transfection-enhancing buffer. The resulting mixture was allowed to sit for 16 hours at 4° C. then dried in vacuo. The wells were then plated with the cells and allowed to culture for 40 hours.

### Example 7

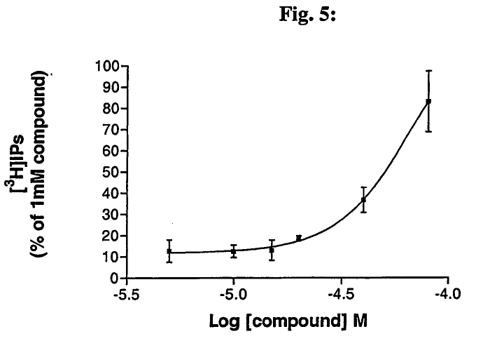

Referring to FIG. 5, depiction of a dose-response curve showing the functional response of transfected cells to glutamate (compound) as measured by PI hydrolysis. Specifically, the MAGEC plated-based, lipid method exemplified in Example 3, spra, was used to co-transfect suitable HEK cells with two nucleic acids, each encoding one of a human GPCR chimeric construct, e.g.mGluR2 and mGluR3 and the promiscuous G protein, Galpha16, into HEK cells.

According to the method, the nucleic acids to be introduced were adhered to the surface of a 24-well tissue culture plate containing a lipid-based transfection reagent (Effectene), an adherence-promoting polymer (PVA) and a transfection-enhancing buffer. The resulting mixture was allowed to sit for 16 hours at 4° C. then dried in vacuo. The wells were then seeded with HEK293 cells and allowed to culture for 24 hours before the start of the assay. The functional assay data points represent mean±SE from three replicates.

### Example 8

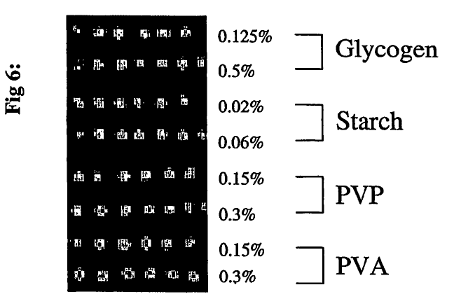

FIG. 6 depicts an image of ³H-labeled drug binding to a cell surface protein HEK293 cells were transfected with a expression vector containing cDNA encoding human calcium subunits-alpha2-delta-1 by the MAGEC slide-based, lipid method (example 2) using varying concentrations of adherence-promoting polymers. Forty hours post-transfection, the cells were rinsed in phosphate-buffered saline, exposed to a ³H-labeled gabapentin that binds to alpha2-delta-1expressed from the transfected DNA, washed and imaged on the Beta-imager. The green and red colored spots indicate the cells within the matrix that are over-expressing the cell surface protein. The blue background indicates binding to the endogenously expressed alpha2-delta-1.

### Example 9

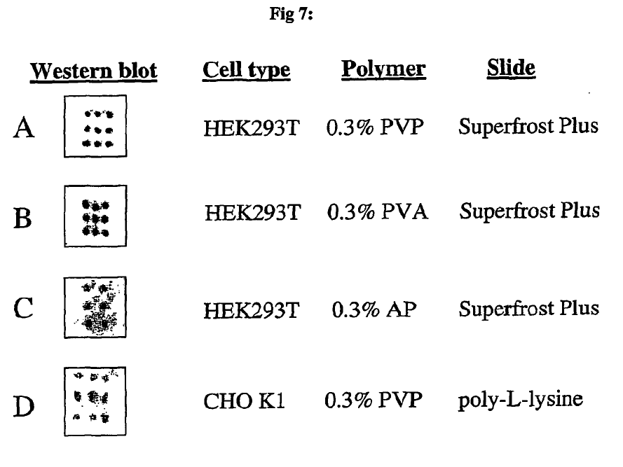

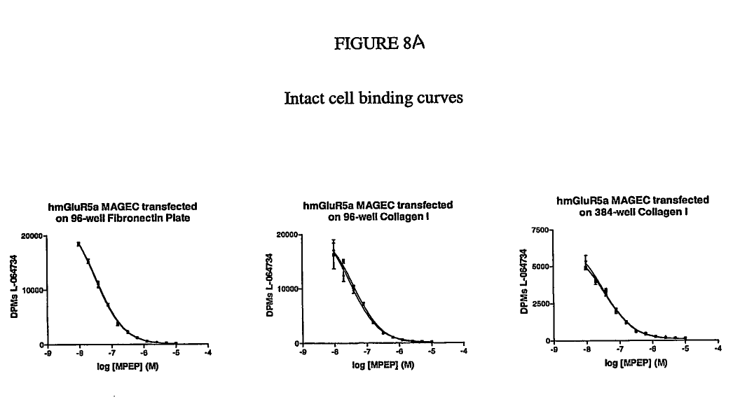

FIG. 7 depicts the results of a Western blot to detect the presence of cells expressing human mGluR5 using an antibody specific for the human mGluR5. Briefly, the slide-based MAGEC protocol of Example 2 was used to transfect HEK293 cells with a heterologous DNA encoding the human metabotropic glutamate receptor subtype 5 (hmGluR5). Forty hours after transfection, the slides were overlaid with a piece of nitrocellulose membrane to promote transfer of the transfected cell monolayer from the slide to the membrane. The membrane was then processed for western blot using a hmGluR5-specific antibody and a positive signal was detected by electro-chemiluminescence using a goat anti-rabbit HRP conjugated antibody. The dark spots in the figure represent clusters of HEK cells expressing hmGluR5. As expected, these dark spots exactly represent the pattern originally printed on the slide. A summary of the different conditions used in A thru D is indicated in the figure.

### Example 10

An MAGEC-based intact cell-binding assay was developed in which HEK293 cells were MAGEC transfected with the human metabotropic subtype 5 cDNA (mGluR5a). The MAGEC-lipid based transfection mixture was deposited to the bottom of a 96-well plate that was coated with either fibronectin, collagen I, or poly-D lysine (PDL) or an 384-well plate coated with collagen I. The mixture was allowed to incubate overnight on the plates then dried in vacuo. BEK293 cells in log-phase growth were seeded to the wells and then allowed to incubate for 40 hours at 37° C. with 6% CO₂. After the 40 hour incubation, the transfected cells were washed with a buffer once (50 mM Tris pH 7.4, 0.9% NaCl) then incubated with 30 nM ³H of labeled compound L, a mGluR5 antagonist, and varying concentration of unlabeled 2-Methyl-6-(phenylethynyl)pyridine hydrochloride (MPEP), wherein compound L is 3-Methoxy-5-(2-Pyridinylethynyl)Pyridine disclosed generally in WO 99/02497, EP 1117403 and EP 00998459, each reference being incorporated by reference herein in its entirety. The cells were then washed twice in cold buffer then trypsinized from the wells and transferred to scintillation vials. Seven ml of scintillation cocktail was added to each vial and counted in a scintillation counter. Each value on the curve represents quadruplicate values. IC₅₀ values obtained from the various intact cell binding assays were very similar to the value obtained from our traditional membrane binding assay (IC₅₀=30 μM). No curves could be generated for the mGluR5a transfected cells plated on PDL-coated plates due to the almost complete cell loss in all wells following the binding assay.

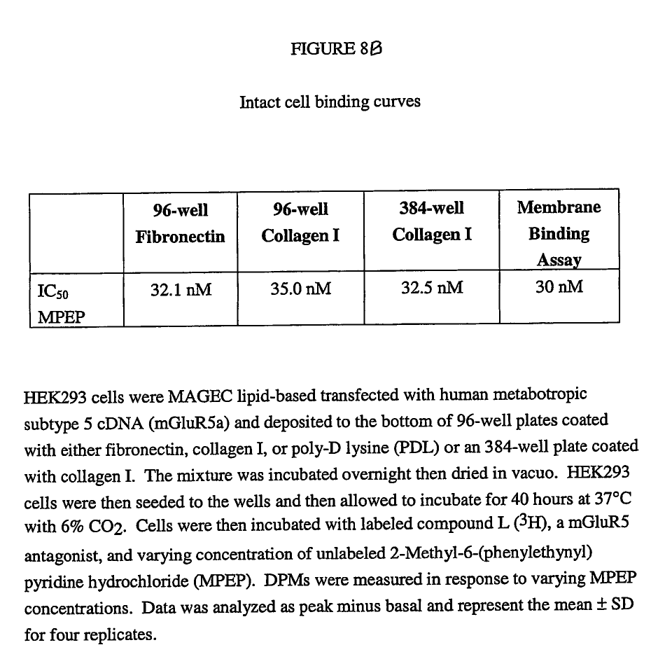

Referring to FIG. 8, it becomes clear that optimal results are obtained when using the proper plate coating. As well, the data demonstrates the unexpectedly superior properties of the methods of the invention over conventional methods utilizing cell adhesion proteins such as PDL. Specifically, the fact that data was unavailable for the PDL coated plates due to cell loss, further corroborates the assertions of the present inventors that conventional assays utilizing cell adhesion proteins such as PDL are attended by numerous drawbacks, chief among which is cell viability or cell health. The data clearly suggests the importance of selecting an appropriate cell adhesion protein, which appears to greatly improves cell adhesion and overall cell viability. This is especially important in binding assays including high throughput assays, which require multiple manipulations. Consequently, if cell viability is compromised, then the assay may fail as it did in the above example.

While this invention has been particularly shown and described with references to preferred embodiments thereof, it will be understood by those skilled in the art that various changes in form and details may be made therein without departing from the scope of the invention encompassed by the appended claims.

1. Matrix analysis of gene expression in cells (MAGEC) is a high throughput gene expression screening system that allows for the indexed introduction, (expression) and analysis of nucleic acids in a host cell comprising:
a) affixing a nucleic acid-containing mixture onto a surface of a suitable solid support medium, wherein the step of affixing comprises depositing a nucleic acid-containing mixture onto the surface of the solid support medium in indexed locations, under conditions favoring formation of a complex between the nucleic acid and the non-proteinaceous, polycationic transfection reagent, wherein the nucleic acid-containing mixture comprises a heterologous nucleic acid contained in a transfection enhancing buffer that is intended to be introduced into suitable host cells in combination with appropriate amounts of an adherence-promoting polymer and a non-proteinaceous, polycationic transfection reagent, wherein the adherence-promoting polymer facilitates adherence of the complex to the surface of the support medium, and the non-proteinaceous, polycationic transfection reagent facilitates condensation of the nucleic acid for uptake by suitable host cells, and allowing the nucleic acid-containing mixture to dry on the surface of the support medium, thereby producing a surface bearing the non-proteinaceous, polycationic transfection reagent-nucleic acid mixture in indexed locations; and
(b) plating the host cells onto the surface bearing the non-proteinaceous, polycationic transfection reagent-nucleic acid mixture in sufficient density and under conditions favoring uptake of the nucleic acid in the mixture by the host cells.

2. The method of claim 1 wherein the affixing step comprises incubating the nucleic acid-containing mixture on a suitable solid support medium between 10-24 hours at 4° C. then the mixtures are dried to the surface in vacuo.

3. The method of claim 1, wherein the nucleic acid is a sense or anti-sense oligonucleotide, RNAi, mRNA, cDNA or genomic DNA, or fragment or portion thereof.

4. The method of claim 1, wherein the adherence-promoting polymer is one of polyvinyl alcohol (PVA), glycogen, amylopectin or methylcellulose.

5. The method of claim 1, wherein the adherence-promoting polymer is a non-proteinaceous reagent that facilitates attachment of the nucleic acid-transfection mixture to the surface of the solid support.

6. The method of claim 1, wherein the nucleic acid is an expression vector and the eukaryotic cells that contain the heterologous nucleic acid are maintained under conditions favoring expression of a gene product encoded by the heterologous nucleic acid.

7. The method of claim 1, wherein the suitable support medium is a multi-well, poly-lysine coated, polystyrene cell culture plate.

8. The method of claim 1, wherein the non-proteinaceous, polycationic transfection reagent is linear polyethylenimine (PEI) reagent.

9. The method of claim 1, where the nucleic acid-containing mixture comprises a sense or anti-sense oligonucleotide.

10. The method of claim 1, further comprising identifying host cells in which a gene product of interest is expressed by the host cell(s), comprising contacting the transfected host cells with a labeled probe having a binding affinity for the gene product of interest under conditions favoring formation of a complex there between, and detecting the formation of the complex as indicating host cells in which the gene product of interest is expressed.

11. The method of claim 10, wherein the gene product of interest is a protein encoded by the heterologous nucleic acid.

12. The method of claim 10, wherein and the labeled probe is an antibody having a binding affinity for the gene product of interest.

13. The method of claim 10, wherein the probe is a peptide or small molecule.

14. The method of claim 1, further comprising identifying host cells in which a gene product of interest is expressed by the host cell(s), comprising contacting the transfected host cells with a labeled probe having a binding affinity for the gene product of interest under conditions favoring disruption of a complex there between, and detecting the disruption of the complex as indicating host cells in which the gene product of interest is expressed.

15. A method of producing a matrix comprising transfected cells, wherein said cells are transfected with a heterologous nucleic acid, comprising:
a) depositing a nucleic acid-containing mixture onto a surface of a solid support medium in indexed locations and allowing the resulting surface bearing the nucleic acid-containing mixture to dry sufficiently that the markings, referred to as nucleic acid-containing markings, remain affixed to the surface under conditions in which the matrix is used, wherein the nucleic acid-containing markings comprise a heterologous nucleic acid in combination with an adherence-promoting polymer and a non-proteinaceous, polycationic transfection reagent;
b) adding cells the surface obtained in a) to produce a surface bearing nucleic acid-containing mixtures in distinct and defined locations and plated cells; and
c) maintaining the surface bearing the nucleic acid containing mixtures and plated cells under conditions favoring the uptake of the nucleic acids by the plated cells, thus producing a matrix of transfected cells that contain the nucleic acids in defined and distinct well location.

16. A matrix produced by the method of claim 15.

17. A gene expression screening system suitable for high throughput screening of nucleic acids in a host cell comprising:
a) affixing a nucleic acid-containing mixture onto a surface of a suitable solid support medium, wherein the step of affixing comprises depositing a nucleic acid-containing mixture onto the surface of the solid support medium in indexed locations, wherein the nucleic acid-containing mixture comprises heterologous nucleic-acid that is intended to be introduced into host cells and appropriate amounts of an adherence-promoting polymer and a lipid-based transfection reagent, and allowing the nucleic acid-containing mixture to dry on the surface; and
b) plating the host cells onto the surface under conditions favoring uptake of the nucleic acid in the nucleic acid-containing mixture by the host cells.

18. (canceled)

19. The gene expression screening system of claim 17, wherein the nucleic acid is contained in an expression vector and the eukaryotic cells that contain the heterologous nucleic acid are maintained under conditions favoring expression of a gene product encoded by the heterologous nucleic acid.

20. The gene expression screening system of claim 17, wherein the vector is of mammalian origin.

21. The gene expression screening system of claim 17, wherein the solid support medium is a positively charged glass slide and the cells are eukaryotic cells.

22. The gene expression screening system of claim 17, wherein the adherence-promoting polymer is one of polyvinyl alcohol (PVA), polyvinyl pyrrolidone (PVP), glycogen or amylopectin.

23. The gene expression screening system of claim 17, wherein the adherence-promoting polymer is a non-proteinaceous reagent that facilitates or allows attachment of the nucleic acid-transfection mixture to the surface of the solid support.

24. The gene expression screening system of claim 17, further comprising identifying host cells in which a gene product of interest is expressed, comprising contacting the transfected host cells on the surface of the suitable support medium with a labeled probe having a binding affinity for the gene product of interest under conditions favoring formation of a complex there between, and detecting the formation of the complex as indicating host cells in which the gene product of interest is expressed.

25. The gene expression screening system of claim 24, wherein the gene product of interest is a protein encoded by the heterologous nucleic acid, and the labeled probe is an antibody having a binding affinity for the gene product of interest.

26. (canceled)

27. (canceled)

28. A method of affixing heterologous nucleic acid molecule to a surface, to produce a matrix of nucleic acids in indexed locations of known or unknown sequence or source for use in a high throughput gene screening system, comprising depositing a heterologous nucleic acid-containing mixture onto the surface in indexed locations and allowing the resulting surface bearing the nucleic acid-containing mixture to dry sufficiently and maintaining the resulting nucleic acid-containing markings under conditions favoring fixation of the markings to the surface under conditions in which the matrix is used.

29. The method of claim 28, wherein the nucleic acid is oligonucleotides, RNAi, mRNA, genomic DNA or cDNA.

30. The method of claim 28, where the nucleic acid containing mixture comprises a heterologous DNA in combination with a adherence-promoting polymer reagent and a lipid-based transfection reagent.

31. The method of claim 28, where the nucleic acid containing mixture comprises a heterologous DNA in combination with a adherence-promoting polymer reagent and a non-proteinaceous, polycationic transfection reagent.

32. A method of producing a matrix of cells transfected with heterologous nucleic acids, comprising:
a) applying a nucleic acid-containing mixture onto a surface of a solid support medium in indexed locations and allowing the resulting surface bearing the nucleic acid-containing mixture to dry sufficiently that the markings, referred to as nucleic acid-containing markings, remain affixed to the surface under conditions in which the matrix is used, wherein the nucleic acid-containing markings include a nucleic acid in combination with a adherence-promoting polymer and a lipid-based transfection reagent;
b) adding cells in an appropriate medium to the surface obtained in a) to produce a slide bearing nucleic acid-containing mixtures in distinct and defined locations and plated cells and
c) maintaining the surface bearing the nucleic acid containing mixtures and plated cells under conditions favoring the uptake of the nucleic acids by the plated cells, thus producing a matrix of transfected cells that contain the nucleic acids in defined and distinct locations.

33. A matrix produced by the method of claim 32.

34. A matrix produced by the method of claim 15.

35. A method of producing a matrix of cells transfected with nucleic acids designated herein as transfected cell matrix, comprising:
a) applying a nucleic acid-containing mixture onto a surface of a solid support medium in indexed locations and allowing the resulting surface bearing the nucleic acid-containing mixture to dry sufficiently that the well of a multi-well plate, referred to as nucleic acid-containing wells, remain affixed to the surface under conditions in which the plates are used, wherein the nucleic acid-containing wells include a nucleic acid in combination with a adherence-promoting polymer and a lipid-based transfection reagent;
b) adding cells in an appropriate medium to the surface obtained in a) to produce a plate-bearing nucleic acid-containing mixtures in distinct and defined locations and plated cells and
c) maintaining the surface bearing the nucleic acid containing mixtures and plated cells under conditions favoring the uptake of the nucleic acids by the plated cells, thus producing a matrix of transfected cells that contain the nucleic acids in defined and distinct well location.

36. A matrix produced by the method of claim 31.

37. (canceled)

38. A method of introducing a heterologous nucleic acid into suitable eukaryotic cells comprising:
a) affixing a nucleic acid-containing mixture onto a surface of a suitable solid support medium, wherein the step of affixing comprises depositing a nucleic acid-containing mixture onto the surface of the solid support medium in indexed locations, under conditions favoring formation of a complex between the nucleic acid and the non-proteinaceous, polycationic transfection reagent, wherein the nucleic acid-containing mixture comprises a heterologous nucleic acid that is intended to be introduced into suitable host cells in combination with appropriate amounts of an adherence-promoting polymer and a non-proteinaceous, polycationic transfection reagent, wherein the adherence-promoting polymer facilitates adherence of the complex to the surface of the support medium, and the non-proteinaceous, polycationic transfection reagent facilitates condensation of the nucleic acid for uptake by suitable host cells, and allowing the nucleic acid-containing mixture to dry on the surface of the support medium, thereby producing a surface bearing the non-proteinaceous, polycationic transfection reagent-nucleic acid mixture in indexed locations; and
(b) plating the host cells onto the surface bearing the non-proteinaceous, polycationic transfection reagent-nucleic acid mixture in sufficient density and under conditions favoring uptake of the nucleic acid in the mixture by the host cells.

39. A transfected cell comprising an expression vector, wherein the expression vector comprises a heterologous nucleic acid and wherein the cell is transfected according to the method of claim 38.

40. A method of screening a compound for competitive binding to a target mammalian receptor on the surface of cells expressing the receptor, the method comprising the following steps:
(a) transforming a host cell according to the method of claim 39 with a heterologous nucleic acid encoding said target receptor, wherein the transfected cells express the target receptor;
(b) assaying the transfected cells with the compound in the presence and in the absence of an agonist for the target receptor; and
(c) determining whether the compound competes with the agonist for binding to the target receptor.

41. The method of claim 40, wherein the compound is detectably-labeled.

42. The method of claim 40, wherein the target receptor agonist is detectably-labeled.

43. The method according to claim 40, wherein the target receptor is previously identified by use of an antibody has a binding affinity for the target receptor.

44. The method of claim 40, wherein the compound that competitively binds to the target receptor is quantitatively characterized by assaying the transfected cells or transfected cell matrix with varying amounts of the compound in the presence of a detectably-labeled target receptor agonist and measuring the extent of competition with agonist binding thereby.

45. A method of screening a compound to determine if the compound is an agonist binding inhibitor of a target mammalian receptor on the surface of cells expressing the receptor, the method comprising the following steps:
(a) transforming a host cell according to the method of claim 39 with a heterologous nucleic acid encoding said target receptor, wherein the transfected cells express the target receptor; and
(b) assaying the transfected cells with the compound in the presence and in the absence of a target receptor agonist to determine whether the compound is capable of inhibiting agonist binding to the target receptor.

46. The method of claim 45, wherein the compound that inhibits target receptor agonist binding is quantitatively characterized by assaying the transfected cells with varying amounts of the compound in the presence of a detectably-labeled target receptor binding agonist and measuring the extent of inhibition of agonist binding thereby.

47. A method of screening a compound for binding a target mammalian receptor on the surface of cells expressing the receptor, the method comprising the following steps:
(a) transforming a host cell according to the method of claim 39 with a heterologous nucleic acid encoding said target receptor, wherein the transfected cells express the target receptor; and
(b) assaying the transfected cells with the compound to determine whether the compound binds to the target receptor.

48. A method for identifying candidate modulators of a target mammalian G protein coupled receptor, which comprises:
incubating a transfected eukaryotic cell wherein a heterologous nucleic acid is introduced according to claim 38, with a test compound, and
detecting a change in the activity of the target mammalian G protein coupled receptor second messenger activity compared to a control cell that either is has not incubated with the compound or does not express the target mammalian G protein coupled receptor, and
relating a change in activity with the ability of the compound to act as a mammalian G protein coupled receptor modulator.

49. A method for identifying agonist or antagonist of a target receptor comprising:
contacting a cell expressing on the surface thereof a mammalian G protein coupled receptor, following transfection according to the method of claim 38, wherein the receptor is associated with a second component capable of providing a detectable signal in response to the binding of a compound to the mammalian receptor, with a compound to be screened under conditions that permit binding of the compound to the receptor; and
determining whether the compound binds to and activates or inhibits the receptor by measuring the level of a signal generated from the interaction of the compound with the receptor.
